(19) United States
(12) Patent Application Publication
(10) Pub. No.: US 2024/0402193 A1
(43) Pub. Date: Dec. 5, 2024

(54) METHODS AND DEVICES FOR DETECTING CEREBROSPINAL FLUID LEAKAGE

(71) Applicant: INTEGRATED NEUROLOGICS LLC, Lancaster, PA (US)

(72) Inventors: Todd FLOWER, Lancaster, PA (US); David MATZILEVICH, Lancaster, PA (US)

(21) Appl. No.: 18/697,160

(22) PCT Filed: Sep. 30, 2022
(86) PCT No.: PCT/US2022/077334

§ 371 Date: Mar. 29, 2024

Related U.S. Application Data
(60) Provisional application No. 63/251,180, filed on Oct. 1, 2021.

(51) Int. Cl. G01N 33/68; G01N 21/78; G01N 33/543

(52) U.S. Cl. CPC G01N 33/6896; G01N 21/78; G01N 33/54388; G01N 33/6878; G01N 2333/4709; G01N 2800/2871

(57) ABSTRACT

Disclosed are methods and devices for detecting the presence or absence of cerebrospinal fluid (CSF) in a biological sample which normally does not contain CSF, in particular to the detection of the presence or absence of a tau protein in the biological sample. Also disclosed are devices and assays for the detection of the presence or absence of a tau protein indicating the presence or absence of CSF in a sample.

### CROSS REFERENCE TO RELATED APPLICATIONS

This application claims priority to U.S. Provisional Application No. 63/251,180, filed 1 Oct. 2021, the entire contents of which are hereby incorporated by reference.

### FIELD

The present disclosure relates generally to methods and devices for detecting the presence or absence of cerebrospinal fluid (CSF) in a biological sample which normally does not contain CSF, in particular the detection of the presence or absence of a tau protein in the biological sample. Also disclosed are devices and assays for the detection of the presence or absence of a tau protein indicating the presence or absence of CSF in a sample.

### BACKGROUND

Cerebrospinal fluid (CSF) leakage occurs when CSF escapes through a small tear or hole in the outermost layer of connective tissue, brain-blood barrier (BBB), or dura mater that surrounds the brain and holds in the CSF. The function of CSF is to cushion the brain and spinal cord and serves as a nutrient delivery and waste removal system for the brain. CSF is manufactured continuously by the choroid plexus located in the ventricles and is absorbed by the bloodstream. In adult humans, about 125 milliliters of CSF is constantly circulated around the brain and the spinal cord, and about another 25 milliliters is in the ventricles for a total of about 150 milliliters of CSF. The total volume of CSF is reabsorbed and replenished 3 to 4 times every 24 hours.

CSF leakage has been traditionally classified as traumatic or non-traumatic. CSF leakage may be spontaneous in the absence of an obvious cause, such as skull base abnormalities or bone erosion related to tumors or hydrocephalus. Spontaneous CSF leakage is often referred to as high pressure leaks when increased intracranial pressure results in the occurrence of a CSF leakage. Additionally, idiopathic, or occult intracranial hypertension (IIH) is increasingly recognized as a cause of spontaneous CSF leakage which can either be released through the nose or through the ears, causing rhinorrhea or otorrhea. Non-traumatic spontaneous leakage can be due to lumbar puncture, a history of epidurals or spinal catheters, head and spinal surgeries, skull-based defects, high pressure intracranial hydrocephalus, untreated or occult intracranial hypertension, underlying and untreated connective tissue diseases, such as Ehlers-Danlos and Marian syndrome, bone spurs along the spine, brain tumors, meningitis (bacterial or viral), brain cysts, brain abscess, or cerebral edema.

The majority of CSF leakage is caused by a traumatic injury (e.g., head trauma or surgery) and occurs predominately in areas of bone or meningeal weakness. There are approximately 3 to 5 million visits annually to emergency rooms in the United States alone and left untreated CSF leakage can lead to morbidity and mortality due to ascending infections leading to meningitis or brain abscess. Therefore, the management of post traumatic CSF leakage remains a clinical challenge. In many instances, CSF rhinorrhea or otorrhea goes undetected either because the patient has a small leak or due to a misdiagnosis such as allergic rhinitis or sinusitis. Indeed, many small leaks occur spontaneously and intermittently which makes a definitive diagnosis problematic.

In traumatic brain injury (TBI), such as a concussive event, there is a rapid increase in intracranial pressure which can result in CSF leakage in the form of rhinorrhea or otorrhea either in a large volume (milliliters) or in very small volumes (microliters). Small volumes or sub-clinical volumes of CSF leakage are extremely difficult to confirm and may result in a lack of treatment. Small CSF leaks such as those which occur in concussion are typically not detectable using imaging techniques such as computed tomography (CT) scans or magnetic resonance imaging (MRI), and small volume leaks may not be detectable using cisternography and can result in life threatening complications.

In the acute setting, diagnostic options for detecting CSF leakage are limited. These include imaging techniques, such as CT scan or MRI, or taking the patient directly to the operating room for management. This is largely guided by the surgeon's discretion and clinical intuition. These imaging modalities are costly, and some expose the patient to radiation. They also fail to detect certain CSF leaks and in particular, small CSF leaks. The alternative, operative management is also an expensive process that exposes the patient to general anesthesia and other perioperative risks. This involves identifying the site of the leak and using either native tissue or biocompatible materials to patch the affected site.

Alternative methods have been developed for the detection of CSF leakage, such as β-2 transferrin electrophoresis or enzyme-linked immunosorbent assay (ELISA), however these assays are expensive and typically require the submission of a sample to a laboratory to perform the assay, delaying receipt of the results and administration of an appropriate treatment when indicated. CSF leakage can also be detected using a nephelometric assay to detect β-trace protein (BTP). While such methods can be rapid and highly sensitive, the BTP nephelometric assay still requires expensive equipment found only in a centralized laboratory and extensive training. As such, these alternative assays are also not suitable for a point-of-care diagnosis due to several practical limitations.

Currently, there are no available tests for inexpensively and non-invasively detecting the presence of a CSF leakage. There remains a need for a quick, reliable, and affordable test to help identify CSF leakage in the outpatient or postoperative settings. Such a test can also be used for a point-of-care diagnosis outside of a hospital or laboratory setting, for example, when a traumatic brain injury, such as a concussion, is suspected to occur. It is often difficult to distinguish normal nasal and otologic secretions from those containing CSF as they may be similar in appearance. This distinction remains important because failure to identify and treat a CSF leakage can result in severe complications, such as meningitis, brainstem herniation, and death. Early diagnosis and proper management are imperative and correlate with a better long-term prognosis of the patients.

### SUMMARY

The present disclosure is directed to methods for the detection of the presence or absence of cerebrospinal fluid (CSF) in a biological sample, which normally does not contain CSF, particularly the detection of the presence or absence of a tau protein in the biological sample. Also disclosed are devices and assays for the detection of the presence or absence of a tau protein indicating the presence or absence of CSF in a sample.

In a first aspect, the disclosure provides a method of detecting a cerebrospinal fluid (CSF) leakage in a subject, said method comprising: a) contacting a rhinorrhea or otorrhea sample from the subject with a first anti-tau antibody and a second anti-tau antibody; and b) detecting the presence or absence of at least one tau protein in the rhinorrhea or otorrhea sample from the subject, wherein detecting the presence of the at least one tau protein in the rhinorrhea or otorrhea sample indicates that the subject has the CSF leakage and detecting the absence of the at least one tau protein in the rhinorrhea or otorrhea sample indicates that the subject does not have the CSF leakage. In some embodiments, the first anti-tau antibody binds to a first epitope of a human tau protein and the second anti-tau antibody binds to a second epitope of the human tau protein, wherein the first epitope comprises the amino acid sequence of QEFEVMEDHAGTY (SEQ ID NO: 1) and the second epitope comprises the amino acid sequence of AAPPGQKGQANA (SEQ ID NO: 2), and the at least one tau protein binds to both the first anti-tau antibody and the second anti-tau antibody. In other embodiments, the first anti-tau antibody and the second anti-tau antibody bind to at least one tau fragment, and the at least one tau fragment comprises at least amino acids 6-168 of SEQ ID NO: 7. In some embodiments, the CSF leakage in the subject is caused by head trauma, spine injury, skull base defects, high pressure intracranial hydrocephalus, or intracranial hypertension. In other embodiments, the CSF leakage in the subject is caused by a traumatic brain injury (TBI), such as a mild TBI or concussion.

In a second aspect, the disclosure provides a method of diagnosing a TBI in a subject in need thereof, said method comprising: a) contacting a biological sample from the subject with a first anti-tau antibody and a second anti-tau antibody; and b) detecting the presence or absence of at least one tau protein in the biological sample from the subject, wherein detecting the presence of the at least one tau protein in the biological sample indicates that the subject has the TBI and detecting the absence of the at least one tau protein in the biological sample indicates that the subject does not have the TBI. In some embodiments, the first anti-tau antibody binds to a first epitope of a human tau protein and the second anti-tau antibody binds to a second epitope of the human tau protein, wherein the first epitope comprises the amino acid sequence of SEQ ID NO: 1 and the second epitope comprises the amino acid sequence of SEQ ID NO: 2, and the at least one tau protein binds to both the first anti-tau antibody and the second anti-tau antibody. In other embodiments, the first anti-tau antibody and the second anti-tau antibody bind to at least one tau fragment, and the at least one tau fragment comprises at least amino acids 6-168 of SEQ ID NO: 7. In some embodiments, the subject is a human suspected of having a TBI, such as a concussion. In some embodiments, the biological sample is obtained from a nose or an ear of the subject. In some embodiments, the biological sample is a biological sample that is suspected of containing CSF but that otherwise normally (e.g., without a traumatic injury) does not contain CSF.

In some embodiments, the first anti-tau antibody used in any one of the disclosed methods is immobilized onto a solid support and the second anti-tau antibody used in any one of the disclosed methods is labeled. In some embodiments, the second anti-tau antibody is labeled by conjugating to a colored latex particle, a gold nanoparticle, or a gold nano-shell. In other embodiments, the second anti-tau antibody is labeled by conjugating to a gold nano-shell having an average diameter of from about 100 nm to about 200 nm. In some embodiments, the solid support forms part of a lateral flow immunoassay device.

In some embodiments, the at least one tau protein detected by any one of the methods disclosed herein is a full-length of tau protein, a fragment of tau protein, and/or an isoform of tau protein having a molecular weight of from about 15 kDa to about 75 kDa. In some embodiments, the at least one tau protein that is detected is a fragment and/or isoform of tau having a molecular weight of about 17 kDa and/or about 30 kDa. In some embodiments, the at least one tau protein to which the first and second anti-tau antibodies bind comprises a first tau fragment or isomer of about 17 kDa, a second tau fragment or isomer of about 25 kDa, a third tau fragment or isomer of about 30 kDa, a fourth tau fragment or isomer of about 35 kDa, a fifth tau fragment or isomer of about 50 kDa, and a sixth tau fragment or isomer of about 75 kDa.

In some embodiments, the rhinorrhea or otorrhea sample or the biological sample from the subject is in a volume of from about 50 microliters to about 200 microliters. In some embodiments, the presence or absence of the at least one tau protein in the rhinorrhea or otorrhea sample or the biological sample from the subject is detected in about 1 minute to about 20 minutes after the contacting step. In some embodiments, the disclosed method has a sensitivity of detecting the at least one tau protein in the biological sample, such as the rhinorrhea or otorrhea sample, at a concentration of from about 0.3 pg/μl to about 1 pg/μl. In other embodiments, the disclosed method has a sensitivity of detecting the at least one tau protein in the biological sample, such as the rhinorrhea or otorrhea sample, at a concentration of from about 0.5 pg/μl to about 0.8 pg/μl. In some embodiments, the disclosed method is not conducted in a hospital or laboratory setting. In some embodiments, the disclosed method is conducted as a point-of-care diagnostic test.

A third aspect is directed to a method of treating a subject diagnosed with or suspected of having a TBI, said method comprising: a) diagnosing the subject as having a TBI by any one of the methods disclosed herein; and b) administering to the subject a therapeutically effective amount of treatment for the TBI. In some embodiments, the treatment for the TBI comprises one or more of: a) resting; b) administering to the subject a therapeutically effective amount of a pain reliever, an anti-seizure drug, a coma-inducing drug, and/or a diuretic; c) surgery; and d) rehabilitation.

In a fourth aspect, the disclosure provides a device comprising: a) a non-immobilized antibody area; b) a lateral flow membrane downstream of and configured for fluid communication with the non-immobilized antibody area; c) a first anti-tau antibody that binds to a first epitope of the human tau protein, wherein the first epitope comprises the amino acid sequence of SEQ ID NO: 1; and d) a second anti-tau antibody that binds to a second epitope of the human tau protein, wherein the second epitope comprises the amino acid sequence of SEQ ID NO: 2. In some embodiments, the first anti-tau antibody and the second anti-tau antibody bind to at least one tau fragment, wherein the at least one tau fragment comprises at least amino acids 6-168 of SEQ ID NO: 7. In some embodiments, the first anti-tau antibody is immobilized at a first location on the lateral flow membrane and the second anti-tau antibody is labeled and located in the non-immobilized antibody area. In some embodiments, the disclosed device further comprises a sample loading area upstream of and configured for fluid communication with the non-immobilized antibody area. In some embodiments, the disclosed device further comprises an absorbent area downstream of and configured for fluid communication with the lateral flow membrane. In some embodiments, the disclosed device further comprises a secondary antibody immobilized at a second location on the lateral flow membrane, wherein the secondary antibody is an anti-immunoglobulin antibody that binds to the second anti-tau antibody. In some embodiments, the secondary antibody is an anti-IgG antibody. In some embodiments, the second location is downstream of the first location.

In some embodiments, the second anti-tau antibody comprised in the disclosed device is labeled by conjugating to a colored latex particle, a gold nanoparticle, or a gold nano-shell. In some embodiments, the second anti-tau antibody is labeled by conjugating to a gold nano-shell having an average diameter of from about 100 nm to about 200 nm.

In some embodiments, the device disclosed herein further comprises a non-specific antibody to reduce or eliminate any potential non-specific binding. In some embodiments, the non-specific antibody is an IgG antibody. In some embodiments, the device disclosed herein further comprises a non-specific blocking agent.

In some embodiments, the sample loading area of the disclosed device comprises a sample loading pad, wherein the sample loading pad comprises cellulose fibers or woven meshes. In some embodiments, the sample loading pad is configured to receive a fluidic sample of about 50 microliters to about 200 microliters. In some embodiments, the non-immobilized antibody area of the disclosed device comprises a non-immobilized antibody pad, and wherein the non-immobilized antibody pad comprises glass fibers, cellulose fibers, surface-treated polyester filters, or surface-treated polypropylene filters. In some embodiments, the lateral flow membrane of the disclosed device is a nitrocellulose membrane. In some embodiments, the absorbent area of the disclosed device comprises an absorbent pad, and wherein the absorbent pad comprises cellulose fibers or woven meshes.

In some embodiments, the disclosed device further comprises a solid substrate supporting the sample loading area, the non-immobilized antibody area, the lateral flow membrane, and the absorbent area. In some embodiments, the disclosed device further comprises a housing. In some embodiments, the disclosed device is in form of a test strip, a dipstick, a flow through device, or a microfluidic device.

In some embodiments, the disclosed device has a sensitivity of detecting at least one tau protein in a sample at a concentration of from about 0.3 pg/μl to about 1 pg/μl. In some embodiments, the disclosed has a sensitivity of detecting at least one tau protein in a sample at a concentration of from about 0.5 pg/μl to about 0.8 pg/μl.

In some embodiments, the disclosed device is configured to change color at the first location on the lateral flow membrane where the first anti-tau antibody is immobilized when the first and second anti-tau antibodies bind to at least one tau protein in a sample. In other embodiments, the disclosed device is configured to change color at the second location on the lateral flow membrane where the secondary antibody is immobilized as a control to show that the labeled second anti-tau antibody has moved from the non-immobilized antibody area to the second location on the lateral flow membrane via lateral flow. In some embodiments, the color change is visible to human eyes. In some embodiments, the color change is detected by a lateral flow reader.

In some embodiments, the disclosed device is a point-of-care diagnostic device. In some embodiments, the disclosed device is an over-the-counter diagnostic device.

In some embodiments, the first anti-tau antibody used in the methods or devices disclosed herein comprises a light chain comprising an amino acid sequence having at least 90% sequence identity to SEQ ID NO: 3 and a heavy chain comprising an amino acid sequence having at least 90% sequence identity to SEQ ID NO: 4. In some embodiments, the first anti-tau antibody used in the methods or devices disclosed herein comprises a light chain comprising the amino acid sequence of SEQ ID NO: 3 and a heavy chain comprising the amino acid sequence of SEQ ID NO: 4. In some embodiments, the second anti-tau antibody used in the methods or devices disclosed herein comprises a light chain comprising an amino acid sequence having at least 90% sequence identity to SEQ ID NO: 5 and a heavy chain comprising an amino acid sequence having at least 90% sequence identity to SEQ ID NO: 6. In some embodiments, the second anti-tau antibody used in the methods or devices disclosed herein comprises a light chain comprising the amino acid sequence of SEQ ID NO: 5 and a heavy chain comprising the amino acid sequence of SEQ ID NO: 6.

In a fifth aspect, the disclosure provides a kit comprising: a) any one of the disclosed devices; and b) a swab comprising an absorbent material configured to absorb cerebrospinal fluids. In some embodiments, the disclosed kit further comprises one or more buffers. In some embodiments, the disclosed kit further comprises instructions for use.

In a sixth aspect, the disclosure provides a method of diagnosing a head trauma, spine injury, skull base defects, high pressure intracranial hydrocephalus, or intracranial hypertension in a subject in need thereof, said method comprising detecting the presence or absence of at least one tau protein in a biological sample from the subject using any one of the devices or kits disclosed herein. In some embodiments, the biological sample is obtained from a nose or an ear of the subject. In some embodiments, the head trauma is a TBI, such as concussion. In some embodiments, the device or the kit is not used in a hospital or laboratory setting and/or is a point-of-care device or kit.

In other embodiments, the disclosure provides method of monitoring progression of recovery from a head trauma, spine injury, skull base defects, high pressure intracranial hydrocephalus, or intracranial hypertension in a subject in need thereof, said method comprising detecting the presence or absence of at least one tau protein in a biological sample from the subject using any one of the devices or kits disclosed herein. In some embodiments, the biological sample is obtained from a nose or an ear of the subject. In some embodiments, the head trauma is a TBI, such as concussion. In some embodiments, the device or the kit is not used in a hospital or laboratory setting and/or is a point-of-care device or kit.

In a seventh aspect, the disclosure further provides a method of treating a subject diagnosed with or suspected of having a head trauma, spine injury, skull base defects, high pressure intracranial hydrocephalus, or intracranial hypertension, said method comprising: a) detecting the presence or absence of at least one tau protein in a biological sample from the subject using any one of the devices or kits disclosed herein; and b) administering to the subject a therapeutically effective amount of treatment for the head trauma, spine injury, skull base defects, high pressure intracranial hydrocephalus, or intracranial hypertension if the presence of the at least one tau protein is detected. In some embodiments, the head trauma is a TBI and the treatment comprises one or more of: a) resting; b) administering to the subject a therapeutically effective amount of a pain reliever, an anti-seizure drug, a coma-inducing drug, and/or a diuretic; c) surgery; and d) rehabilitation. In some embodiments, the biological sample is obtained from a nose or an ear of the subject. In some embodiments, the head trauma is a concussion. In some embodiments, the device or the kit is not used in a hospital or laboratory setting and/or is a point-of-care device or kit.

### BRIEF DESCRIPTION OF THE DRAWINGS

The accompanying drawings, which are incorporated in and constitute a part of this specification, illustrate certain embodiments, and together with the written description, serve to explain certain principles of the methods and devices disclosed herein.

FIG. 1 depicts a schematic representation of the full-length human tau protein (1-441) displaying the currently identified proteolytic cleavage sites.

FIG. 2 depicts a schematic representation of the various epitopes or antibody binding sites of the human tau protein tested in Example 1. The blue lines represent the positions on the tau protein where each specific antibody binds. The numbers above or below each respective blue line identify the amino acid residues in the epitopes that are recognized by each individual antibody. The red lines represent two antibodies that bind to the interior of the tau protein, but their exact epitope sequences have not been elucidated.

FIG. 3A-3J depict immunoprecipitation of the tau protein and/or fragments thereof in CSF samples using a panel of antibodies followed by the Western blot analysis. Each panel consists of 5 lanes for the Western blot analysis. Lane 1: Molecular weight standard; Lane 2: Purified recombinant tau protein as a positive control; Lanes 3-5: different pooled donor CSF samples. FIG. 3A: The anti-tau antibody that binds to epitope 1-150 was immobilized for the immunoprecipitation and the anti-tau antibody that binds to epitope 157-168 was used to probe the Western blot. FIG. 3B: The anti-tau antibody that binds to epitope 157-168 was immobilized for the immunoprecipitation and the anti-tau antibody that binds to epitope 210-230 was used to probe the Western blot. FIG. 3C: The anti-tau antibody that binds to epitope 157-168 was immobilized for the immunoprecipitation and the anti-tau antibody that binds to epitope 404-441 was used to probe the Western blot. FIG. 3D: The anti-tau antibody that binds to epitope 1-100 was immobilized for the immunoprecipitation and the anti-tau antibody that binds to epitope 210-230 was used to probe the Western blot. FIG. 3E: The anti-tau antibody that binds to epitope 6-18 was immobilized for the immunoprecipitation and the anti-tau antibody that binds to epitope 404-441 was used to probe the Western blot. FIG. 3F: The anti-tau antibody that binds to epitope 1-100 was immobilized for the immunoprecipitation and the anti-tau antibody that binds to epitope 210-230 was used to probe the Western blot. FIG. 3G: The anti-tau antibody that bind to epitope 6-18 was immobilized for the immunoprecipitation and the anti-tau antibody that binds to epitope 157-168 was used to probe the Western blot.

FIG. 3H: The anti-tau antibody that binds to the epitope 6-18 was immobilized for the immunoprecipitation and the anti-tau antibody that binds to epitope 185-195 was used to probe the Western blot. FIG. 3I: The anti-tau antibody that binds to epitope 6-18 was immobilized for the immunoprecipitation and the anti-tau antibody that binds to the epitope 210-230 was used to probe the Western blot. FIG. 3J: The two anti-tau antibodies that bind to the interior of the tau protein with their corresponding epitope sequence unknown.

FIG. 4A-4D depict densitometry of five tau proteolytic fragment bands from selected Western blot analysis in CSF samples. For all panels the sample types are as follows: yellow bar: TBI CSF sample (lumbar CSF), and green bar: repetitive TBI CSF sample (rhinorrhea CSF). FIG. 4A: Densitometry results for tau proteolytic fragments observed on the Western blot analysis for the antibody pair recognizing epitopes 6-18 and 157-168. FIG. 4B: Densitometry results for tau proteolytic fragments observed on the Western blot analysis for the antibody pair recognizing epitopes 1-100 and 157-168 (the 5th band was not detected with this antibody pair). FIG. 4C: Densitometry results for tau proteolytic fragments observed on the Western blot analysis for the antibody pair recognizing epitopes 1-100 and 210-230. FIG. 4D: Densitometry results for tau proteolytic fragments observed on the Western blot analysis for the antibody pair recognizing epitopes 157-168 and 185-195 (the 5th band was not detected with this antibody pair).

FIG. 5 depicts a general lateral flow immunoassay design, which is composed of the following elements: a sample loading pad (where the sample containing the analyte is applied), a non-immobilized antibody pad, or conjugate pad, (where the conjugated antibody has been dried down), lateral flow membrane with immobilized antibodies (where the analyte and bound conjugated antibody are captured to generate the test line), and absorbent pad (which facilities the flow of the fluid through the device). The components of the strip are usually fixed to a solid substrate, such as an inert backing material.

FIG. 6 depicts an exemplary design of a device according to one embodiment of the disclosure. In this design, the non-immobilized antibody pad, or conjugate pad, contains the antibody recognizing the tau epitope 157-168 (AB 157-168) conjugated to gold nano-shells and the lateral flow membrane contains the antibody recognizing the tau epitope 6-18 (AB 6-18) immobilized on the test line and the secondary antibody, such as an anti-IgG antibody, immobilized on the control line.

FIG. 7A-7B depict the mechanism of action of the CSF lateral flow immunoassay according to one embodiment of the disclosure. FIG. 7A represents a schematic representation of the lateral flow immunoassay mechanism of action. Top: the sample containing tau proteins (“Analyte”) is deposited on the sample loading pad and migrates towards the conjugate pad containing a first anti-tau antibody conjugated to a label (“conjugated antibody”). Middle and bottom: the conjugated antibody binds to the tau proteins (middle), if present in the sample, and (bottom) migrates to the test line, where the bound tau proteins are captured with a second anti-tau antibody (“capture antibody”). The conjugated antibody is generally present in an excess amount compared to the tau proteins and therefore, some of the conjugated antibody will not be captured at the test line and will continue to flow toward the control line. The control line contains an immobilized secondary antibody that binds to the conjugated antibody (e.g., species specific anti-immunoglobulin antibody). The binding of the conjugated antibody to the immobilized secondary antibody provides a positive control to show that the conjugated antibody has moved from the conjugate pad to the control line via lateral flow. FIG. 7B: An exemplary read out of the CSF lateral flow immunoassay according to one embodiment of the disclosure.

FIG. 8A-8C depict lateral flow immunoassay results on various clinical samples using the anti-tau antibodies AB 6-18 and AB 157-168 (FIG. 8A) or a selected group of anti-tau antibodies that recognize various other epitopes other than epitopes 6-18 and 157-168 (FIG. 8B). Line 1: the test line; Line 2: the control line. FIG. 8C depicts that a clear, visible test line can only be seen in Lanes 1, 3, and 5 where the antibody that binds to epitopes 157-168 was used as the conjugated antibody, but not Lanes 2, 4, and 6 wherein the antibody that binds to epitopes 6-18 was used as the conjugated antibody.

FIG. 9 depicts the component positioning of an exemplary lateral flow immunoassay device according to one embodiment of the disclosure.

FIG. 10 depicts comparison of glycosylation of a recombinant antibody that binds to an epitope comprising amino acid residues 6-18 of SEQ ID NO: 7 that is produced in CHO cells (CHO 6-18) (left) and a mouse monoclonal antibody that binds to an epitope comprising amino acid residues 6-18 of SEQ ID NO: 7 (6-18 mouse mAb) (right). The antibody was either untreated (lane 2) or treated (lane 3) with the glycosidase PNGase F. A molecular weight shift in the heavy chain of the CHO 6-18 antibody (left, lane 3) was observed, indicating the removal of the N-linked oligosaccharides. No molecular weight shift was observed with the 6-18 mouse mAb (right), suggesting that the 6-18 mouse mAb is not glycosylated or is glycosylated to a lesser degree than the CHO 6-18 antibody.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the disclosure, and should not be interpreted as a limitation of the scope of the disclosure.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of +20%, +10%, +5%, +1%, +0.9%, +0.8%, +0.7%, +0.6%, +0.5%, +0.4%, +0.3%, +0.2% or +0.1% from the specified value as such variations are appropriate to perform the disclosed methods and/or to make and use the disclosed devices. When “about” is present before a series of numbers or a range, it is understood that “about” can modify each of the numbers in the series or range.

The term “absence of the at least one tau protein [in a sample]” is used herein to mean that the sample is devoid of all detectable tau proteins, including the full-length tau protein, any differentially spliced isoforms of tau, and tau fragments.

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “antibody” or “antibodies” as used in this disclosure refers to an immunoglobulin or an antigen-binding fragment thereof. As will be understood by those in the art, the immunological binding reagents encompassed by the term “antibody” or “antibodies” extend to all antibodies from all species, and antigen binding fragments thereof and include, unless otherwise specified, polyclonal, monoclonal, monospecific, bispecific, polyspecific, humanized, human, camelised, mouse, non-human primates, single-chain, chimeric, synthetic, recombinant, hybrid, mutated, CDR-grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda.

The term “antigen” refers to any substance that is capable of generating an immune response (e.g., the production of antibodies).

The terms “antigen-binding domain” and “antigen-binding fragment” refer to a part of an antibody molecule that comprises amino acids responsible for the specific binding between antibody and antigen. For certain antigens, the antigen-binding domain or antigen-binding fragment of an antibody molecule may only bind to a part of the antigen. The part of the antigen that is specifically recognized and bound by the antibody is referred to as the “epitope” or “antigenic determinant.” Antigen-binding domains and antigen-binding fragments include Fab (Fragment antigen-binding); a F(ab′)₂ fragment, a bivalent fragment having two Fab fragments linked by a disulfide bridge at the hinge region; Fv fragment; a single chain Fv fragment (scFv) see e.g., Bird et al. (1988) Science 242:423-426; and Huston et al. (1988) Proc. Natl. Acad. Sci. USA 85:5879-5883); a Fd fragment having the two V_{H} and C_{H}1 domains; dAb (Ward et al., (1989) Nature 341:544-546), and other antibody fragments that retain antigen-binding function. The Fab fragment has V_{H}-C_{H} and V_{L}-C_{L} domains covalently linked by a disulfide bond between the constant regions. The Fᵥ fragment is smaller and has V_{H} and V_{L} domains non-covalently linked. To overcome the tendency of non-covalently linked domains to dissociate, a scF, can be constructed. The scF, contains a flexible polypeptide that links (1) the C-terminus of V_{H} to the N-terminus of V_{L}, or (2) the C-terminus of V_{L} to the N-terminus of V_{H}. These antibody fragments are obtained using conventional techniques known to those with skill in the art, and the fragments are evaluated for function in the same manner as are intact antibodies.

The term “at least” prior to a number or series of numbers (e.g., “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

As used herein, the terms “binds” or “binding” refer to the interaction between an antibody, or an antigen-binding fragment, and an antigen, or an antigenic fragment.

The term “biological sample” refers to a sample of biological tissue, cells, or fluid that, in a healthy and/or pathological state, may contain CSF from a subject that has a CSF leakage. Illustrative samples include, but not limited to, oral fluid samples, nasal fluid samples, aural fluid samples, and ear drainage samples, and the like. Although the sample is typically taken from a human patient, the assays can be used to detect CSF in samples from any mammal, such as dogs, cats, sheep, cattle, and pigs, etc. The sample may be pretreated as necessary by dilution in an appropriate buffer solution or concentrated, if desired. Any of known standard aqueous buffer solutions, such as phosphate, Tris, or the like, at or near physiological pH can be used and the term sample is intended to include pre-treated samples as well as acute samples.

The term “diagnosing” or “diagnosis” as used herein refers to the use of information (e.g., antibody binding or data from tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals sharing common nucleotide sequences, symptoms, signs, family histories, or other data relevant to consideration of a patient's health status.

The term “downstream” when used with reference to a lateral flow device indicates that the downstream location is further along the lateral flow device in the direction of fluid flow (capillary flow) than another location. Thus, if location B is downstream from location A, then fluid flowing through the lateral flow device will reach location A before reaching location B.

The term “epitope” or “antigenic determinant” refers to the part of an antigen that is specifically recognized and bound by a particular antibody variable region.

The term “fragment,” as used herein in reference to a protein, refers to a polypeptide that has an amino-terminal and/or carboxy-terminal deletion as compared to the native, full-length protein. Fragments typically are at least 4 amino acids long, preferably at least 20 amino acids long, usually at least 50 amino acids long or longer, and may span the portion of the full-length protein required for intermolecular binding with its various ligands and/or substrates.

The term “identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988).

Typical methods to determine identity are designed to give the largest match between the sequences tested. Methods to determine identity and similarity are codified in publicly available computer programs. Typical computer program methods to determine identity and similarity between two sequences include, but are not limited to, the GCG program package (Devereux, J., et al., Nucleic Acids Research 12 (1): 387 (1984)), BLASTP, BLASTN, and FASTA (Atschul, S. F. et al., J. Molec. Biol. 215:403-410 (1990). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The term “immunoassay” refers to any assay that uses at least one specific antibody for the detection and/or quantification of an antigen. Immunoassays include, but not limited to, rapid strip tests, Western blots, enzyme-linked immunosorbent assays (ELISAs), radio-immunoassays, and immunofluorescence assays and any other antigen-antibody reactions including, for example, “flocculation” (i.e., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen), “complement fixation” (i.e., the use of complement in an antibody-antigen reaction method), and other methods commonly used in serology, immunology, immunocytochemistry, immunohistochemistry, and related fields.

The term “in need thereof” means that the subject has been identified or suspected as having a need for the particular method or treatment. In some embodiments, the identification can be by any means of diagnosis or observation. In any of the methods described herein, the subject can be in need thereof. In some embodiments, the subject in need thereof is a human suspected of having a traumatic brain injury (TBI). In some embodiments, the subject in need thereof is a human diagnosed with TBI. In some embodiments, the subject in need thereof is a human seeking treatment for TBI. In some embodiments, the subject in need thereof is a human undergoing treatment for TBI.

As used herein, the term “in some embodiments” refers to embodiments of all aspects of the disclosure, unless the context clearly indicates otherwise.

The term “kit” refers to a combination of reagents and/or apparatus, which facilitates sample analysis. In some embodiments, a kit may further include one or more apparatus to facilitate sample harvesting. In some embodiments, a kit may further include one or more reagents for sample processing. In some embodiments, a kit may further include one or more written instructions.

The term “labeled” as used herein in reference to anti-tau antibody refers to attaching to the anti-tau antibody any substance which is capable of producing a signal that is detectable by visual or instrumental means. Various substances suitable for labeling an anti-tau antibody in the present disclosure can include chromogens, catalysts, fluorescent compounds (such as, for example, fluorescein, phycobiliprotein, rhodamine), chemiluminescent compounds, radioactive elements, colloidal metallic (such as gold), non-metallic (such as selenium) and dye particles, enzymes, enzyme substrates, and organic polymer latex particles, liposomes or other vesicles containing such signal producing substances, and the like. Examples of enzymes that can be used for labeling an anti-tau antibody include, but not limited to, phosphatases and peroxidases, such as alkaline phosphatase and horseradish peroxidase which are used in conjunction with enzyme substrates, such as nitro blue tetrazolium, 3,5′,5,5′-tetranitrobenzidine, 4-methoxy-1-naphthol, 4-chloro-1-naphthol, 5-bromo-4-chloro-3-indolyl phosphate, chemiluminescent enzyme substrates such as the dioxetanes. In some embodiments, the anti-tau antibody is labeled using a colored latex particle, a gold nanoparticle, or a gold nano-shell.

The term “point-of-care” refers to the point in time when healthcare products and services are delivered to patients at the time of care. A “point-of-care” test or method, also called bedside testing, refers to a medical diagnostic test or method that can be performed at or near the point of care—that is, at the time and place of patient care. In the diagnostic setting, this means that the diagnosis occurs at the time and place where the test is administered to the patient. For example, with a point-of-care embodiment in the context of the methods disclosed herein, a sample may be obtained from the patient and tested using the methods and/or devices disclosed herein without having to send the sample to a different location for testing. In this way, the result of the test can be provided to the patient at the same location where the test was administered, typically within minutes, or in even less time. A “point-of-care” testing contrasts with testing that is wholly or mostly confined to a medical laboratory and often entails collecting a sample and sending the sample away from the point of care location and then waiting hours or days to learn the results, during which time care must be withheld or administered without the desired diagnostic result.

The term “protein” refers to a polymer of amino acids, peptide nucleic acids (PNAs) or mimetics, of no specific length and to all fragments, isoforms, variants, derivatives and modifications (glycosylation, phosphorylation, post-translational modifications, etc.) thereof.

The term “sample” is used herein in the broadest sense and can be obtained from any source in the body. A sample can encompass fluids, solids and/or tissues. In some embodiments, a sample can include one or more of the following fluids: aural fluid, nasal fluid, or ear drainage. A sample can also include other fluids, such as serous fluid, urine, saliva, tears, blood, plasma, and serum. A “rhinorrhea sample” refers to a sample containing nasal fluid or drainage. An “otorrhea sample” refers to a sample containing aural fluid and/or ear drainage.

The terms “subject,” “patient,” and “individual” are used interchangeably herein to refer to any mammalian subject for whom diagnosis or therapy is desired, particularly humans, hospitalized or not.

The term “substrate” refers to any rigid or semi-rigid support to which molecules (e.g., nucleic acids, polypeptides, mimetics) may be bound. Examples of substrates include, but not limited to, membranes, filters, chips, slides, wafers, fibers, magnetic, or nonmagnetic beads, gels, capillaries or other tubing, plates, polymers, and microparticles with a variety of surface forms including wells, trenches, pins, channels, and pores.

The term “tau,” “tau protein” or “human tau protein” refers to a group of highly soluble protein isoforms produced by alternative splicing from the gene MAPT (microtubule-associated protein tau) and fragments thereof, including proteolytic fragments of tau. Tau can exist in phosphorylated forms (see, e.g., Goedert, Proc. Natl. Acad. Sci. U.S.A. 1988, 85:4051-4055; Goedert, EMBO J., 1989, 8:393-399; Lee, Neuron, 1989, 2:1615-1624; Goedert, Neuron, 1989, 3:519-526; Andreadis, Biochemistry, 1992, 31:10626-10633) and has been reported to have a role in stabilizing microtubules, particularly in the central nervous system. Unless otherwise apparent from the context, reference to tau or tau protein means a natural human form of tau including all isoforms and fragments thereof irrespective of whether a posttranslational modification (e.g., phosphorylation, glycation, or acetylation) is present.

The term “treat,” “treated,” or “treating” refers to therapeutic treatment and/or prophylactic or preventative measures wherein the object is to prevent or slow down (lessen) an undesired physiological condition, disorder, or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

As used herein, the term “therapeutic” means an agent utilized to treat, combat, ameliorate, prevent, or improve an unwanted condition or disease of a patient.

A “therapeutically effective amount” of a treatment is a predetermined amount calculated to achieve the desired effect, i.e., to treat, combat, ameliorate, prevent, or improve one or more symptoms of a viral infection. The activity contemplated by the present methods includes both medical therapeutic and/or prophylactic treatment, as appropriate. The specific dose of a compound administered according to the present disclosure to obtain therapeutic and/or prophylactic effects will, of course, be determined by the circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds for treating TBI according to the disclosure is typically an amount such that when it is administered in a physiologically tolerable excipient composition, it is sufficient to achieve an effective systemic concentration or local concentration in the tissue.

The term “traumatic brain injury” or “TBI” refers to a neurotrauma caused by a mechanical force that is applied to the head. TBI is a heterogenous neurological disorder—it ranges from penetrating injury, focal contusion, different forms of hematoma (subdural, epidural) to diffuse injury to single or repetitive mild TBI. Mild TBI, often synonymous with concussion, typically affects the frontal and temporal lobes of the brain which are associated with executive function, learning, and memory (Stuss et al., Curr. Opin. Neurol., 2011, 24 (6): 584-589). In some embodiments, the TBI is a severe, penetrating brain injury. In some embodiments, the TBI is a mild TBI. In some embodiments, the TBI is a concussion.

### Detection of Cerebrospinal Fluid (CSF) Leakage

The disclosure relates to methods for detection and diagnosis of cerebrospinal fluid (CSF) leakage and associated conditions using antibodies that bind to a tau protein, including proteolytic tau fragments. CSF is a clear liquid similar in appearance to water, and in composition to plasma. The brain and spinal cord are rendered buoyant and protected by the CSF. Clinical, surgical, and accidental events may cause CSF to breach its physiologic barriers. CSF leakage may occur with the placement of needles and catheters for anesthesia and analgesia, trauma, skull fractures, intracranial surgical procedures, infection, hydrocephalus, congenital malformations, neoplasms, and spontaneous rhinorrhea and otorrhea.

The human tau protein is a member of the microtubule-associated family of proteins which are expressed exclusively in the central nervous system (CNS) and particularly, in unmyelinated axons and cortical inter-neurons (Trojanowski et al., J. Histochem. Cytochem., 1989, 37:209-215; Sivanandam et al., Neurosci. Biobehav. Rev., 2012, 36:1376-1381). The primary functions of the tau protein include the stabilization of microtubules, which are important cytoskeleton scaffolds for the cells and support cellular trafficking (Cleveland et al., J. Mol. Biol., 1977, 166 (2): 227-247) and the coordinated movement of molecules along the microtubule (Mandelkow et al., Cold Spring Harbor Perspect. Med., 2012, 2 (7): a006247; Iqbal et al., Mol. Neurobiol., 1991, 5 (2-4): 399-410; Khatoon et al., FEBS Lett., 1994, 351 (1): 80-84).

The human tau protein occurs mainly in the axons of the CNS and consists largely of six major isoforms generated by alternative splicing (Goedert et al., EMBO J., 1989, 8:393-399; Goedert et al., Neuron, 1989, 3 (4): 519-526). The molecular weights of the six major isoforms (without post-translational modifications) are approximately 67 kDA, 62 kDa, 59 kDa, 54 kDa, 52 kDa and 48 kDa, respectively. As discussed below, however, tau proteins undergo extensive post-translational modifications and, thus, the molecular weights of these full-length tau isomers and fragments thereof may be higher than these reported values. The tau isomers differ by the presence or absence of two near-amino-terminal inserts of 29 residues each, encoded by exons 2 and 3, and by one of the repeats (R2, 31 residues) in the carboxy-terminal half. Hence, the six isoforms are from 352 to 441 amino acids in length and contain either zero, one, or two amino-terminal inserts (0N, 1N, or 2N) and either three or four microtubule binding repeats (3R or 4R). Different names are in use, derived from inserts/repeats, number of residues, or clone names, as summarized in Table 1. The overall structure of tau is composed of an amino-terminal projection domain, followed by microtubule binding repeats, and a short carboxyl-terminal tail sequence (Schweers et al., J. Biol. Chem., 1994, 269 (30): 24290-24297).

TABLE 1
Isoforms of human tau proteins (Mandelkow et al., Cold
Spring Harb. Perspect. Med., 2012, 2(7): a006247).

| Clone | Inserts/Repeats | Number of amino acids (AA) |
|---|---|---|
| htau40 | 2N4R | 441 |
| htau39 | 2N3R | 410 |
| htau34 | 1N4R | 412 |
| htau37 | 1N3R | 381 |
| htau24 | 0N4R | 383 |
| htau23 | 0N3R | 352 |
| big Tau | 2N4R + exon 4a | 695 |

The main function of the tau protein is to modulate the stability of axonal microtubules. Tau is not normally present in dendrites and is active primarily in the distal portions of axons where it provides microtubule stabilization but also flexibility as needed. Tau generates a partially stable, but still dynamic state in microtubules important for the dynamics of axonal growth cones and effective axonal transport. Some studies have shown that tau can interact, either directly or indirectly, with actin and affect actin polymerization as well as the interaction of actin filaments with microtubules (Yamauchi et al., Biochem. Biophys. Res. Commun., 1993, 190 (3): 710-715; Selden et al., J. Biol. Chem., 1983, 258 (11): 7064-7070; Henriquez et al., Cell Biochem. Funct., 1995, 13 (4): 239-250).

The tau protein undergoes multiple post-translational modifications, such as glycosylation, non-enzymatic glycosylation (glycation), and phosphorylation. Phosphorylation represents the major post-translational modification of the tau protein, and its biological activity is regulated by the degree of its phosphorylation. Phosphorylation has been reported on approximately 30 sites in the normal tau protein (Billingsley et al., Biochem. J., 1997, 323 (Pt3): 577-591). These phosphorylation events can control the normal biological functions of tau, as well as its pathological functions, such as the ability to self-assemble into neuronal filaments found in neurodegenerative diseases. It is currently believed that the hyperphosphorylation of tau results in their dissociation from the microtubules where the protein has a greater propensity to aggregate.

Hyperphosphorylated tau aggregates are often detected after TBI (Dale et al., J. Beurol. Neurosurg. Psychiatry, 1991, 54:116-118; Mckee et al., J. Neuropathol. Exp. Neurol., 2010, 69:918-929; McKee et al., Brain, 2013, 136:43-64; Omalu et al., Neurosurgery, 2011, 69:173-183; Blennow et al., Nat. Rev. Dis. Primers, 2016, 2:16084). In addition, loss of axonal microtubules resulting from direct or indirect traumatic injury to the CNS axons is a common feature of head trauma (Povlishock et al., J. Neurotrauma, 1995, 12:555-564). This loss of axonal microtubules following injury would be expected to release intracellular microtubule binding proteins, such as tau, into the extracellular space where they would be detectable in the CSF (Povlishock et al., Acta. Neurochir. Suppl., 1996, 66:81-86; Segal et al., J. Inherit. Metab. Dis., 1993, 16:617-638). This has been demonstrated experimentally where tau protein levels were elevated >1,000-fold in CSF samples from TBI patients when compared with controls (Zemlan et al., J. Neurochemistry, 1999, 72 (2): 741-750).

This same study also demonstrated that tau found in the CSF of TBI was proteolyzed into different fragments ranging from 30-50 kDa. Given that tau is an intraneuronal non-released/secreted protein, the level of tau in the CSF of subjects free of axonal injury would be expected to be low or non-existent, making tau a good candidate for a specific CSF biomarker.

However, one complication with using tau as a CSF biomarker is tau proteolysis. As demonstrated in Zemlan et al. (J. Neurochemistry, 1999, 72 (2): 741-750), tau found in the CSF of TBI was proteolyzed into different fragments ranging from 30-50 kDa. Tau proteolysis has been the subject of a considerable amount of research interest because of its involvement with age-related neurodegenerative diseases, or tauopathies, mainly through calpain activation (Lee et al., Prog. Mol. Biol. Transl. Sci., 2012, 107:263-293), a protease that is upregulated in numerous neurological conditions. Calpain has been reported to be induced after TBI (Mondello et al., J. Neurotrauma, 2010, 27:1203-1213) and calpain degradation of tau has been reported to produce a 35 kDa fragment and a 17 kDa fragment of tau that can contribute to neurotoxic events within the brain cells (Park et al., J. Neurosci., 2005, 25:5365-5375).

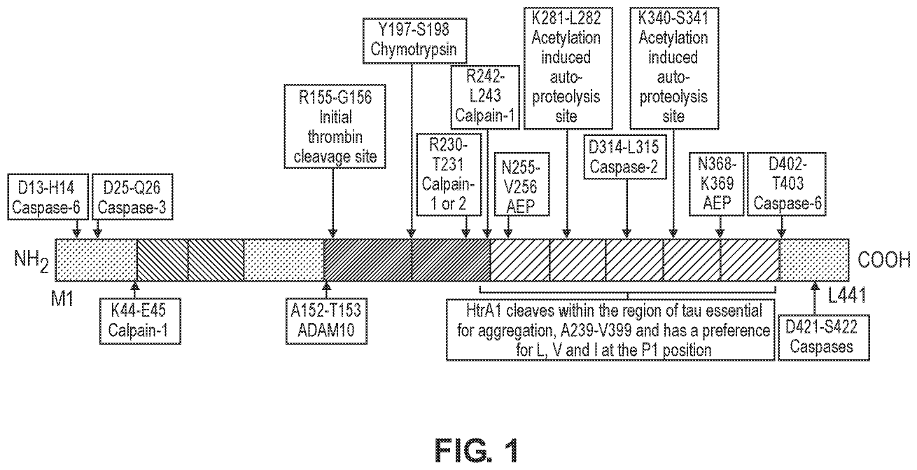

The tau protein is susceptible to various proteolytic modifications at various sites along the protein as shown in FIG. 1. The majority of the proteolytic modifications have been deduced through research on Alzheimer's disease. Proteases currently known to be involved in the proteolytic cleavage of tau are: caspase-6 (Horowitz et al., J. Neuroscience, 2004, 24:7895-7902; Guo et al., Am. J. Pathol., 2004, 165:523-531), calpain-1 (Yang et al., Eur. J. Biochem., 1995, 233:9-17), a disintegrin and metalloprotease 10 (ADAM10) (Henriksen et al., PloS One, 2013, 8 (5): e64990), initial thrombin cleavage site (Arai et al., J. Biol. Chem., 2005, 280:5145-5153), chymotrypsin (Steiner et al., EMBO J., 1990, 9:3539-3544), human high temperature requirement serine protease A1 (HtrA1) (Tennstaedt et al., J. Biol. Chem., 2012, 287:20931-20941), asparagine endopeptidase (AEP) (Zhang et al., Nat. Med., 2014, 20:1254-1262), acetylation-induced auto-proteolysis sites (Cohen et al., PLOS One, 2016, 11 (7): e0158470), caspases-7 and caspases-8 (Gamblin et al., Proc. Natl. Acad. Sci. USA, 2003, 100:10032-10037), and cleavage sites for which the protease is yet to be identified (Derisbourg et al., Sci. Rep., 2015, 5:1-10; Novak et al., EMBO J., 1993, 12:365-370). Numerous other proteases known to proteolyze the tau protein are upregulated following TBI, including caspase-1 (Liu et al., J. Neuroinflammation, 2018, 15 (1): 48), delta-secretase (Wu et al., Prog. Neurobiol., 2020, 185:101730), caspase-3 (Glushakova et al., J. Neurotrauma, 2018, 35 (1): 157-173), and lysosomal proteases (Park et al., J. Neurotrauma, 2015, 32 (19): 1449-1457).

Proteolytic cleavage of a target protein, such as tau, can disrupt epitopes and prevent efficient antibody binding, leading to low or no detection of the target protein and/or a failed diagnostic test. Furthermore, CSF leakage samples, such as a rhinorrhea sample or an otorrhea sample, typically contain a small amount of CSF fluid with small amounts of tau protein. Thus, the proteolysis of the tau protein in CSF makes it challenging to detect tau in CSF leakage samples, such as a rhinorrhea sample or an otorrhea sample, particularly with sufficient sensitivity to effectively employ tau as a diagnostic biomarker.

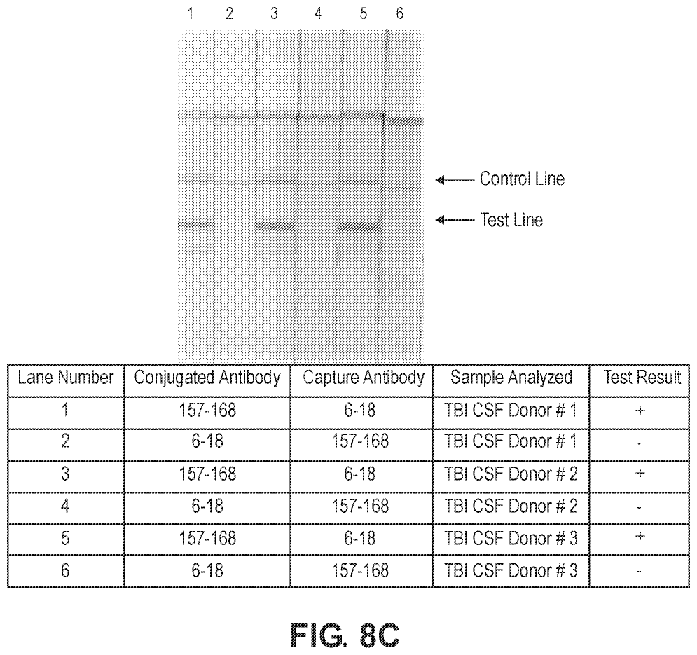

It has been discovered that a specific pair of antibodies directed against two specific tau epitopes can be combined to generate a highly sensitive immunoassay for detecting the tau protein in CSF leakage samples. Extensive research with multiple antibody pairs recognizing different tau epitopes showed that only the combination of a first antibody that binds to a first tau epitope comprising the amino acid sequence of SEQ ID NO: 1 and a second antibody that binds to a second tau epitope comprising the amino acid sequence of SEQ ID NO: 2 resulted in the highly sensitive detection of the tau protein in CSF leakage samples (FIG. 8A). If one of the antibodies (i.e., the first or the second antibody) was combined with an antibody that bound to a different tau epitope, the assay did not reliably detect tau protein (FIG. 8B). Thus, the specific combination of the first and second antibodies was required to accurately detect tau protein in CSF leakage samples with high sensitivity, such that the immunoassay could be developed as a point-of-care assay. Without intending to be bound by any theory, it appears that this specific combination of antibodies detects more proteolytic fragments of tau and/or detects those proteolytic fragments with higher sensitivity than other antibody combinations. Furthermore, the immunoassay was designed to be used as a point-of-care diagnostic assay that does not require training or professional expertise to use and provides accurate, sensitive results within minutes, or even shorter.

Accordingly, in some embodiments, provided herein is a method for the detection of a cerebrospinal fluid (CSF) leakage in a subject, the method comprising detecting the presence or absence of at least one tau protein in a biological sample suspected of containing CSF, wherein the at least one tau protein comprises a first epitope comprising the amino acid sequence of SEQ ID NO: 1 and a second epitope comprising the amino acid sequence of SEQ ID NO: 2, and wherein detecting the presence of the at least one tau protein in the biological sample indicates that the subject has the CSF leakage and detecting the absence of the at least one tau protein in the biological sample indicates that the subject does not have the CSF leakage. In some embodiments, the biological sample suspected of containing CSF is a rhinorrhea sample from the subject. In other embodiments, the biological sample suspected of containing CSF is an otorrhea sample from the subject.

In some embodiments, the CSF leakage in the subject is caused by head trauma. In some embodiments, the CSF leakage in the subject is caused by spine injury. In some embodiments, the CSF leakage in the subject is caused by skull base defects. In some embodiments, the CSF leakage in the subject is caused by high pressure intracranial hydrocephalus. In some embodiments, the CSF leakage in the subject is caused by intracranial hypertension.

In some embodiments, the subject is diagnosed with or suspected of having a head trauma. In some embodiments, the subject is diagnosed with or suspected of having a TBI. In some embodiments, the subject is diagnosed with or suspected of having a mild TBI. In some embodiments, the subject is diagnosed with or suspected of having a concussion. In some embodiments, the subject has spontaneous rhinorrhea. In some embodiments, the subject has spontaneous otorrhea. In some embodiments, the subject is undergoing, or has undergone, head trauma. In some embodiments, the subject is undergoing, or has undergone, surgery. In some embodiments, the subject is undergoing, or has undergone, neural blockade.

Also provided herein, in some embodiments, is a method of diagnosing a TBI in a subject in need thereof, said method comprising detecting the presence or absence of at least one tau protein in a biological sample from the subject, wherein the at least one tau protein comprises a first epitope comprising the amino acid sequence of SEQ ID NO: 1 and a second epitope comprising the amino acid sequence of SEQ ID NO: 2, and wherein detecting the presence of the at least one tau protein in the biological sample indicates that the subject has the TBI and detecting the absence of the at least one tau protein in the biological sample indicates that the subject does not have the TBI. In some embodiments, a biological sample is obtained from a nose of a subject or is a rhinorrhea sample. In other embodiments, a biological sample is obtained from an ear of a subject or is an otorrhea sample.

In some embodiments, the subject is a human suspected of having a TBI. In some embodiments, the subject is a human suspected of having a mild TBI. In some embodiments, the subject is a human suspected of having a concussion.

The at least one tau protein comprising the first epitope comprising the amino acid sequence of SEQ ID NO: 1 and the second epitope comprising the amino acid sequence of SEQ ID NO: 2 can be detected by anti-tau antibodies that specifically bind to these two epitopes. The at least one tau protein may be a full-length tau protein and/or a fragment thereof, such as a proteolytic tau fragment. Thus, in some embodiments, the disclosed method comprises contacting a biological sample, such as a rhinorrhea or otorrhea sample, of the subject with a first anti-tau antibody that binds to the first epitope and a second anti-tau antibody that binds to the second epitope, and detecting the presence or absence of the at least one tau protein in the biological sample, such as the rhinorrhea or otorrhea sample, of the subject, wherein the at least one tau protein binds to both the first anti-tau antibody and the second anti-tau antibody. In other embodiments, the disclosed method comprises contacting a biological sample, such as a rhinorrhea or otorrhea sample, of the subject with a first anti-tau antibody and a second anti-tau antibody, wherein the first and second anti-tau antibodies bind to at least one tau fragment, and detecting the presence or absence of the at least one tau fragment in biological sample, such as the rhinorrhea or otorrhea sample, of the subject, wherein the at least one tau fragment comprises at least amino acids 6-168 of SEQ ID NO: 7.

The amino acid sequence of SEQ ID NO: 7 corresponds to the full-length human tau protein clone htau40 provided in Table 1, which is the Tau-441 isoform containing two amino-terminal inserts (2N) and four microtubule binding repeats (4R). It has 441 amino acids with the following sequence:

(SEQ ID NO: 7)
MAEPRQEFEVMEDHAGTYGLGDRKDQGGYTMHQDQEGDTDAGLKESPLQ
TPTEDGSEEPGSETSDAKSTPTAEDVTAPLVDEGAPGKQAAAQPHTEIP
EGTTAEEAGIGDTPSLEDEAAGHVTQARMVSKSKDGTGSDDKKAKGADG
KTKIATPRGAAPPGQKGQANATRIPAKTPPAPKTPPSSGEPPKSGDRSG
YSSPGSPGTPGSRSRTPSLPTPPTREPKKVAVVRTPPKSPSSAKSRLQT
APVPMPDLKNVKSKIGSTENLKHQPGGGKVQIINKKLDLSNVQSKCGSK
DNIKHVPGGGSVQIVYKPVDLSKVTSKCGSLGNIHHKPGGGQVEVKSEK
LDFKDRVQSKIGSLDNITHVPGGGNKKIETHKLTFRENAKAKTDHGAEI
VYKSPVVSGDTSPRHLSNVSSTGSIDMVDSPQLATLADEVSASLAKQGL.

The first epitope corresponds to amino acids 6-18 of SEQ ID NO: 7 and comprises the amino acid sequence of QEFEVMEDHAGTY (SEQ ID NO: 1). The second epitope corresponds to amino acids 157-168 of SEQ ID NO: 7 and comprising the sequence of AAPPGQKGQANA (SEQ ID NO: 2). These two epitopes are italicized in the sequence of SEQ ID NO: 7 provided above.

Any anti-tau antibodies that specifically bind to these two epitopes can be used in any of the disclosed methods. Examples of anti-tau antibodies that specifically bind to the first epitope of SEQ ID NO: 1 include, but not limited to, anti-Tau, 6-18 antibody (clone Tau 12) available at BIOLEGEND® and MAGIC™ anti-tau (aa 6-18) monoclonal antibody available at CREATIVE DIAGNOSITCS® or the CHO 6-18 antibody described herein. In some embodiments, the anti-tau antibody that specifically binds to the first epitope of SEQ ID NO: 1 is a mouse monoclonal antibody. In some embodiments, the anti-tau antibody that specifically binds to the first epitope of SEQ ID NO: 1 is a recombinant antibody produced in an animal cell. In some embodiments, the anti-tau antibody that specifically binds to the first epitope of SEQ ID NO: 1 is a recombinant antibody produced in a CHO cell. As shown in the Examples, antibodies produced in animal cells, such as CHO cells, can have unique glycosylation patterns. In certain embodiments, the anti-tau antibody that specifically binds to the first epitope of SEQ ID NO: 1 can comprise a light chain comprising the following sequence:

(SEQ ID NO: 3)
DVLMTQTPLSLAVNLGDQASLSCRSSQTILHSNGNTYLEWYLQKPGQSP
RLLIYKVSKRFSGVPDRFSGSGSGTDFTLKISRVEADDLGIYYCFQGSL
VPWAFGGGTKLEIKRADAAPTVSIFPPSSEQLTSGGASVVCFLNNFYPK
DINVKWKIDGSERQNGVLNSWTDQDSKDSTYSMSSTLTLTKDEYERHNS
YTCEATHKTSTSPIVKSFNRNEC,

and a heavy chain comprising the following sequence:

(SEQ ID NO: 4)
EVQLVESGEDLVKPGGSLKLSCVASGFAFSSYGMSWVRQTPDMRLEWVA
TISSSGSRTYFPDSVKGRLTISRDNDKNILYLQMSSLRSEDTAMYYCTI
TWDGAMDYWGRGISVTVSSAKTTPPSVYPLAPGSAAQTNSMVTLGCLVK
GYFPEPVTVTWNSGSLSSGVHTFPAVLQSDLYTLSSSVTVPSSTWPSET
VTCNVAHPASSTKVDKKIVPRDCGCKPCICTVPEVSSVFIFPPKPKDVL
TITLTPKVTCVVVDISKDDPEVQFSWFVDDVEVHTAQTQPREEQFNSTF
RSVSELPIMHQDWLNGKEFKCRVNSAAFPAPIEKTISKTKGRPKAPQVY
TIPPPKEQMAKDKVSLTCMITDFFPEDITVEWQWNGQPAENYKNTQPIM
DTDGSYFVYSKLNVQKSNWEAGNTFTCSVLHEGLHNHHTEKSLSHSPGK.

Non-limiting examples of anti-tau antibodies that specifically bind to the second epitope of SEQ ID NO: 2 is the anti-Tau, 157-168 antibody (clone 2G9.F10) available at BIOLEGEND® and the 157-168 CHO antibody described herein. In some embodiments, the anti-tau antibody that specifically binds to the second epitope of SEQ ID NO: 2 is a mouse monoclonal antibody. In some embodiments, the anti-tau antibody that specifically binds to the second epitope of SEQ ID NO: 2 is a recombinant antibody produced in an animal cell. In some embodiments, the anti-tau antibody that specifically binds to the second epitope of SEQ ID NO: 2 is a recombinant antibody produced in a CHO cell. As shown in the Examples, antibodies produced in animal cells, such as CHO cells, can have unique glycosylation patterns. In certain embodiments, the anti-tau antibody that specifically binds to the second epitope of SEQ ID NO: 2 can comprise a light chain comprising the following sequence:

(SEQ ID NO: 5)
DVVMTQTPLTLSVTIGQPASISCKSSQSLLYSNGKTYLNWLLQRPGQSP
KRLIYLVSKLDSGVPDRFTGSGSGTDFTLKISRVEAEDLGVYYCVQGTH
FPLTFGAGTKLELKRADAAPTVSIFPPSSEQLTSGGASVVCFLNNFYPK
DINVKWKIDGSERQNGVLNSWTDQDSKDSTYSMSSTLTLTKDEYERHNS
YTCEATHKTSTSPIVKSFNRNE,

and a heavy chain comprising the following sequence:

(SEQ ID NO: 6)
QVTLKESGPGILQPSQTLSLTCSFSGFSLSTFGMGVGWFRQPSGKGLEW
LAHIWWDDDKYYNPALKSRLTISKDTSKNQVFLKIANVDTADTATYYCA
RNYDYDVFAYWGQGTLVTVSAAKTTPPSVYPLAPGSAAQTNSMVTLGCL
VKGYFPEPVTVTWNSGSLSSGVHTFPAVLQSDLYTLSSSVTVPSSTWPS
QTVTCNVAHPASSTKVDKKIVPRDCGCKPCICTVPEVSSVFIFPPKPKD
VLTITLTPKVTCVVVDISKDDPEVQFSWFVDDVEVHTAQTKPREEQINS
TFRSVSELPIMHQDWLNGKEFKCRVNSAAFPAPIEKTISKTKGRPKAPQ
VYTIPPPKEQMAKDKVSLTCMITNFFPEDITVEWQWNGQPAENYKNTQP
IMDTDGSYFVYSKLNVQKSNWEAGNTFTCSVLHEGLHNHHTEKSLSHSP
GK.

Accordingly, in some embodiments, the anti-tau antibody that specifically binds to the first epitope of SEQ ID NO: 1 comprises a light chain comprising an amino acid sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 3. In some embodiments, the anti-tau antibody that specifically binds to the first epitope of SEQ ID NO: 1 comprises a heavy chain comprising an amino acid sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 4. In some embodiments, the anti-tau antibody that specifically binds to the first epitope of SEQ ID NO: 1 comprises a light chain comprising an amino acid sequence having at least about 80% sequence identity to SEQ ID NO: 3 and a heavy chain comprising an amino acid sequence having at least about 80% sequence identity to SEQ ID NO: 4. In some embodiments, the anti-tau antibody that specifically binds to the first epitope of SEQ ID NO: 1 comprises a light chain comprising an amino acid sequence having at least about 85% sequence identity to SEQ ID NO: 3 and a heavy chain comprising an amino acid sequence having at least about 85% sequence identity to SEQ ID NO: 4. In some embodiments, the anti-tau antibody that specifically binds to the first epitope of SEQ ID NO: 1 comprises a light chain comprising an amino acid sequence having at least about 90% sequence identity to SEQ ID NO: 3 and a heavy chain comprising an amino acid sequence having at least about 90% sequence identity to SEQ ID NO: 4. In some embodiments, the anti-tau antibody that specifically binds to the first epitope of SEQ ID NO: 1 comprises a light chain comprising an amino acid sequence having at least about 95% sequence identity to SEQ ID NO: 3 and a heavy chain comprising an amino acid sequence having at least about 95% sequence identity to SEQ ID NO: 4. In some embodiments, the anti-tau antibody that specifically binds to the first epitope of SEQ ID NO: 1 comprises a light chain comprising the amino acid sequence of SEQ ID NO: 3 and a heavy chain comprising the amino acid sequence of SEQ ID NO: 4.

In other embodiments, the anti-tau antibody that specifically binds to the second epitope of SEQ ID NO: 2 comprises a light chain comprising an amino acid sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 5. In some embodiments, the anti-tau antibody that specifically binds to the second epitope of SEQ ID NO: 2 comprises a heavy chain comprising an amino acid sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 6. In some embodiments, the anti-tau antibody that specifically binds to the second epitope of SEQ ID NO: 2 comprises a light chain comprising an amino acid sequence having at least about 80% sequence identity to SEQ ID NO: 5 and a heavy chain comprising an amino acid sequence having at least about 80% sequence identity to SEQ ID NO: 6. In some embodiments, the anti-tau antibody that specifically binds to the second epitope of SEQ ID NO: 2 comprises a light chain comprising an amino acid sequence having at least about 85% sequence identity to SEQ ID NO: 5 and a heavy chain comprising an amino acid sequence having at least about 85% sequence identity to SEQ ID NO: 6. In some embodiments, the anti-tau antibody that specifically binds to the second epitope of SEQ ID NO: 2 comprises a light chain comprising an amino acid sequence having at least about 90% sequence identity to SEQ ID NO: 5 and a heavy chain comprising an amino acid sequence having at least about 90% sequence identity to SEQ ID NO: 6. In some embodiments, the anti-tau antibody that specifically binds to the second epitope of SEQ ID NO: 2 comprises a light chain comprising an amino acid sequence having at least about 95% sequence identity to SEQ ID NO: 5 and a heavy chain comprising an amino acid sequence having at least about 95% sequence identity to SEQ ID NO: 6. In some embodiments, the anti-tau antibody that specifically binds to the second epitope of SEQ ID NO: 2 comprises a light chain comprising the amino acid sequence of SEQ ID NO: 5 and a heavy chain comprising the amino acid sequence of SEQ ID NO: 6.

In some embodiments, the anti-tau antibody that specifically binds to the first epitope of SEQ ID NO: 1 comprises a light chain comprising an amino acid sequence having the complementarity determining region 1 (CDR1), CDR2 and CDR3 of SEQ ID NO: 3. In some embodiments, the anti-tau antibody that specifically binds to the first epitope of SEQ ID NO: 1 comprises a heavy chain comprising an amino acid sequence having the CDR1, CDR2 and CDR3 of SEQ ID NO: 4. In some embodiments, the anti-tau antibody that specifically binds to the first epitope of SEQ ID NO: 1 comprises a light chain comprising an amino acid sequence having the CDR1, CDR2 and CDR3 of SEQ ID NO: 3 and a heavy chain comprising an amino acid sequence having the CDR1, CDR2 and CDR3 of SEQ ID NO: 4.

In some embodiments, the anti-tau antibody that specifically binds to the second epitope of SEQ ID NO: 2 comprises a light chain comprising an amino acid sequence having the CDR1, CDR2 and CDR3 of SEQ ID NO: 5. In some embodiments, the anti-tau antibody that specifically binds to the first epitope of SEQ ID NO: 2 comprises a heavy chain comprising an amino acid sequence having the CDR1, CDR2 and CDR3 of SEQ ID NO: 6. In some embodiments, the anti-tau antibody that specifically binds to the first epitope of SEQ ID NO: 2 comprises a light chain comprising an amino acid sequence having the CDR1, CDR2 and CDR3 of SEQ ID NO: 5 and a heavy chain comprising an amino acid sequence having the CDR1, CDR2 and CDR3 of SEQ ID NO: 6.

The disclosed methods can detect the presence of full-length human tau or proteolytic fragments thereof, if present, in a biological sample from a subject. Due to the proteolysis of the tau protein, the tau fragments present in the sample can be of different length and, thus, have different molecular weights. In some embodiments, the disclosed methods detect the presence of at least one tau protein that is the full-length tau protein, a differentially spliced isoform of the tau protein, or a fragment of tau having a molecular weight of from about 10 kDa to about 80 kDa. In some embodiments, the disclosed methods detect the presence of at least one tau protein that is a fragment or isoform of tau having a molecular weight of from about 15 kDa to about 75 kDa. In some embodiments, the disclosed methods detect the presence of at least one tau protein that is a fragment or isoform of tau having a molecular weight of from about 20 kDa to about 60 kDa. In some embodiments, the disclosed methods detect the presence of at least one tau protein that is a fragment or isoform of tau having a molecular weight of from about 25 kDa to about 50 kDa. In some embodiments, the disclosed methods detect the presence of at least one tau protein that is a fragment or isoform of tau having a molecular weight of about 15, about 17 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, or about 80 kDa. In some embodiments, the disclosed methods detect the presence of at least two tau fragments or isoforms of different molecular weight. In some embodiments, the disclosed methods detect the presence of at least three tau fragments or isoforms of different molecular weight. In some embodiments, the disclosed methods detect the presence of at least four tau fragments or isoforms of different molecular weight. In some embodiments, the disclosed methods detect the presence of at least five tau fragments or isoforms of different molecular weight. In some embodiments, the disclosed methods detect the presence of at least six tau fragments or isoforms of different molecular weight. In some embodiments, the disclosed methods detect the presence of one to six tau fragments or isoforms selected from a 17 kDa fragment or isoform, a 25 kDa fragment or isoform, a 30 kDa fragment or isoform, a 35 kDa fragment or isoform, a 50 kDa fragment or isoform, and a 75 kDa fragment or isoform. In some embodiments, the disclosed methods detect the presence of a first tau fragment or isoform of about 17 kDa and a second tau fragment or isoform of about 30 kDa. In some embodiments, the disclosed methods detect the presence of a first tau fragment or isoform of about 17 kDa, a second tau fragment or isoform of about 25 kDa, and a third tau fragment or isoform of about 30 kDa. In some embodiments, the disclosed methods detect the presence of a first tau fragment or isoform of about 17 kDa, a second tau fragment or isoform of about 25 kDa, a third tau fragment or isoform of about 30 kDa, and a fourth tau fragment or isoform of about 35 kDa. In some embodiments, the disclosed methods detect the presence of a first tau fragment or isoform of about 17 kDa, a second tau fragment or isoform of about 25 kDa, a third tau fragment or isoform of about 30 kDa, a fourth tau fragment or isoform of about 35 kDa, and a fifth tau fragment or isoform of about 50 kDa. In some embodiments, the disclosed methods detect the presence of a first tau fragment or isoform of about 17 kDa, a second tau fragment or isoform of about 25 kDa, a third tau fragment or isoform of about 30 kDa, a fourth tau fragment or isoform of about 35 kDa, a fifth tau fragment or isoform of about 50 kDa, and a sixth tau fragment or isoform of about 75 kDa. In some embodiments, the disclosed methods also detect the full-length tau protein in any isoform.

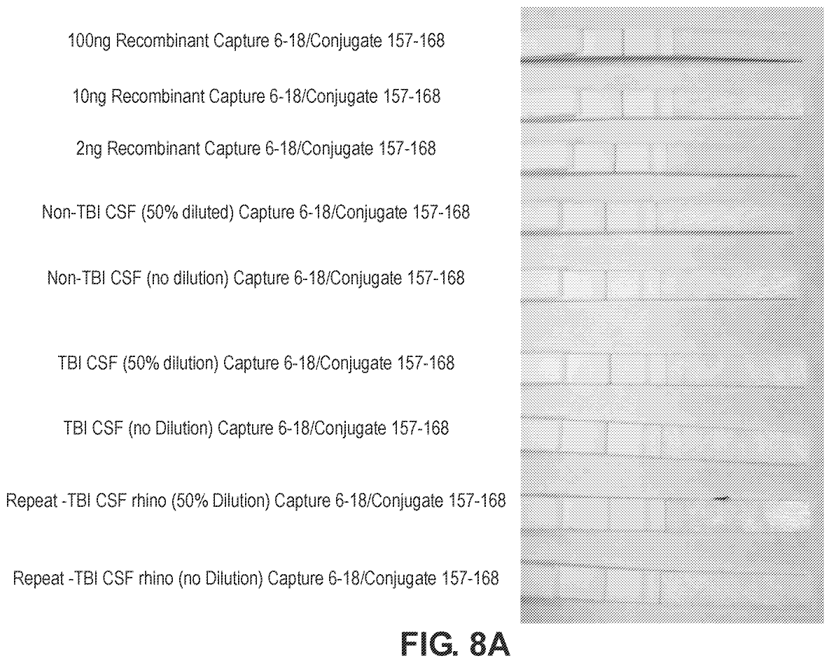

The disclosed methods can be performed as a lateral flow immunoassay, an Enzyme-Linked Immunosorbent Assays (ELISAs), as well as various immune-electrophoretic assay. In some embodiments, the methods are performed as a lateral flow immunoassay using a lateral flow immunoassay device. In such embodiments, either the first anti-tau antibody recognizing the first epitope of SEQ ID NO: 1 or the second anti-tau antibody recognizing the second epitope of SEQ ID NO: 2 can be immobilized onto a solid support, and the other is labeled. In a preferred embodiment, the first anti-tau antibody recognizing the first epitope of SEQ ID NO: 1 is immobilized onto a solid support and the second anti-tau antibody recognizing the second epitope of SEQ ID NO: 2 is labeled, as illustrated, for example, in the lateral flow immunoassay device shown in FIG. 6. In other embodiments, the second anti-tau antibody recognizing the second epitope of SEQ ID NO: 2 is immobilized onto a solid support and the first anti-tau antibody recognizing the first epitope of SEQ ID NO: 1 is labeled. In some embodiments, the solid support onto which the anti-tau antibody is immobilized forms part of a lateral flow immunoassay device.

The anti-tau antibody can be labeled with any substance using any method known in the art so long as the substance used can produce a signal that is detectable by visual or instrumental means. Various substances suitable for labeling an anti-tau antibody in the present disclosure can include chromogens, catalysts, fluorescent compounds (such as, for example, fluorescein, phycobiliprotein, rhodamine), chemiluminescent compounds, radioactive elements, colloidal metallic (such as gold), non-metallic (such as selenium) and dye particles, enzymes, enzyme substrates, and organic polymer latex particles, liposomes or other vesicles containing such signal producing substances, and the like. Examples of enzymes that can be used for labeling an anti-tau antibody include, but not limited to, phosphatases and peroxidases, such as alkaline phosphatase and horseradish peroxidase which are used in conjunction with enzyme substrates, such as nitro blue tetrazolium, 3,5′,5,5′-tetranitrobenzidine, 4-methoxy-1-naphthol, 4-chloro-1-naphthol, 5-bromo-4-chloro-3-indolyl phosphate, chemiluminescent enzyme substrates such as the dioxetanes. In some embodiments, the anti-tau antibody is labeled by conjugating to an enzyme. In some embodiments, the anti-tau antibody is labeled by conjugating to a colored latex particle. In some embodiments, the anti-tau antibody is labeled by conjugating to a gold nanoparticle. In some embodiments, the anti-tau antibody is labeled by conjugating to a gold nano-shell.

Gold nano-shells are surface plasmon resonant (SPR) nanoparticles consisting of a nanoscale silica core surrounded by an ultra-thin gold shell. Changing the ratio of the core diameter and the shell thickness tunes the absorption and scattering properties of the nano-shells across the visible and near-infrared (NIR) regions of the electromagnetic spectrum. Increasing the size of the silica core and decreasing the thickness of the gold shell cause the plasmon resonance to shift toward the NIR. Gold nano-shells and their uses thereof are disclosed in, for instance, U.S. Pat. Nos. 6,344,272, 6,685,986, 6,699,724, and 7,371,457, the contents of which are incorporated by reference herein. In some embodiments, the anti-tau antibody is labeled by conjugating to a gold nano-shell having an average diameter of from about 50 nm to about 500 nm. In some embodiments, the anti-tau antibody is labeled by conjugating to a gold nano-shell having an average diameter of from about 50 nm to about 400 nm. In some embodiments, the anti-tau antibody is labeled by conjugating to a gold nano-shell having an average diameter of from about 75 nm to about 300 nm. In some embodiments, the anti-tau antibody is labeled by conjugating to a gold nano-shell having an average diameter of from about 100 nm to about 200 nm. In some embodiments, the anti-tau antibody is labeled by conjugating to a gold nano-shell having an average diameter of about 50 nm, about 75 nm, about 100 nm, about 150 nm, about 200 nm, about 250 nm, about 300 nm, about 350 nm, about 400, about 450 nm, or about 500 nm.

The methods disclosed herein can detect small amounts of tau protein in biological samples, such as a sample suspected of containing CSF leakage, with a high sensitivity that was not previously possible. In some embodiments, the methods have a sensitivity of detecting the at least one tau protein in the biological sample, such as the rhinorrhea or otorrhea sample, of the subject at a concentration of from about 0.1 pg/μl to about 10 pg/μl. In some embodiments, the methods have a sensitivity of detecting the at least one tau protein in the biological sample, such as the rhinorrhea or otorrhea sample, of the subject at a concentration of from about 0.1 pg/μl to about 5 pg/μl. In some embodiments, the methods have a sensitivity of detecting the at least one tau protein in the biological sample, such as the rhinorrhea or otorrhea sample, of the subject at a concentration of from about 0.1 pg/μl to about 4 pg/μl. In some embodiments, the methods have a sensitivity of detecting the at least one tau protein in the biological sample, such as the rhinorrhea or otorrhea sample, of the subject at a concentration of from about 0.1 pg/μl to about 3 pg/μl. In some embodiments, the methods have a sensitivity of detecting the at least one tau protein in the biological sample, such as the rhinorrhea or otorrhea sample, of the subject at a concentration of from about 0.1 pg/μl to about 2 pg/μl. In some embodiments, the methods have a sensitivity of detecting the at least one tau protein in the biological sample, such as the rhinorrhea or otorrhea sample, of the subject at a concentration of from about 0.1 pg/μl to about 1 pg/μl. In some embodiments, the methods have a sensitivity of detecting the at least one tau protein in the biological sample, such as the rhinorrhea or otorrhea sample, of the subject at a concentration of from about 0.2 pg/μl to about 1 pg/μl. In some embodiments, the methods have a sensitivity of detecting the at least one tau protein in the biological sample, such as the rhinorrhea or otorrhea sample, of the subject at a concentration of from about 0.3 pg/μl to about 1 pg/μl. In some embodiments, the methods have a sensitivity of detecting the at least one tau protein in the biological sample, such as the rhinorrhea or otorrhea sample, of the subject at a concentration of from about 0.5 pg/μl to about 1 pg/μl. In some embodiments, the methods have a sensitivity of detecting the at least one tau protein in the biological sample, such as the rhinorrhea or otorrhea sample, of the subject at a concentration of from about 0.5 pg/μl to about 0.8 pg/μl.

The sensitivity and specificity of the methods disclosed herein can be further increased by using a non-specific antibody to reduce or eliminate any potential non-specific binding. Any non-specific antibody, such as non-specific mouse IgG, can be used. Examples of such non-specific antibodies include, but are not limited to, the blocking reagents, such as mouse IgG and, heterophilic blocking reagents (HBRs, e.g., HBR-11) manufactured by Scantibodies Laboratory, Inc. (Santee, CA). In some embodiments, the non-specific antibody is an IgG antibody. In some embodiments, the IgG is a non-specific mouse IgG. A non-specific IgG from a species other than mouse may be used. In other embodiments, the non-specific antibody is a heterophilic blocking reagent, such as HBR-11. In some embodiments, the non-specific antibody (e.g., mouse IgG) is added into the storage buffer for either or both of the first and second anti-tau antibodies. In some embodiments, the non-specific antibody (e.g., mouse IgG) is added into the sample before the contacting step. In some embodiments, the non-specific antibody (e.g., mouse IgG) is added at the same time when the sample is in contact with the first and second anti-tau antibodies. In some embodiments, the non-specific antibody (e.g., mouse IgG) is added in the sample pad during the manufacture of the device.

When in use, the concentration of the non-specific antibody should be sufficient to block any potential non-specific binding between the first and second anti-tau proteins and any other non-tau antigens in the biological sample. In some embodiments, the concentration of the non-specific antibody used in the methods disclosed herein is from about 1 μg/mL to about 5.0 mg/mL, such as from about 10 μg/mL to about 4.5 mg/mL, from about 15 μg/mL to about 4.0 mg/mL, from about 20 μg/mL to about 3.5 mg/mL, from about 25 μg/mL to about 4.0 mg/mL, or from about 50 g/mL to about 5.0 mg/mL. In some embodiments, the concentration of the non-specific antibody is from about 0.1 mg/mL to about 5.0 mg/mL. In some embodiments, the concentration of the non-specific antibody is from about 0.5 mg/mL to about 4.5 mg/mL. In some embodiments, the concentration of the non-specific antibody is from about 0.5 mg/mL to about 4.0 mg/mL. In some embodiments, the concentration of the non-specific antibody is from about 0.5 mg/mL to about 3.5 mg/mL. In some embodiments, the concentration of the non-specific antibody is from about 0.5 mg/mL to about 3.0 mg/mL. In some embodiments, the concentration of the non-specific antibody is from about 0.5 mg/ml to about 2.5 mg/mL. In some embodiments, the concentration of the non-specific antibody is from about 0.5 mg/mL to about 2.0 mg/mL. In some embodiments, the concentration of the non-specific antibody is from about 0.5 mg/mL to about 1.5 mg/mL. In some embodiments, the concentration of the non-specific antibody is at least about 1, 5, 10, 15, 16, 17, 18, 19, 20, 25, 30, 35, 40, 50, 75, or 100 μg/mL. In some embodiments, the concentration of the non-specific antibody is at least about 0.2, 0.3, 0.4, 0.5, 0.6, 0.7, 0.8, 0.9 or 1.0 mg/mL. In some embodiments, the concentration of the non-specific antibody, such as a generic IgG, is at least about 15 μg/mL. In some embodiments, the concentration of the non-specific antibody, such as a generic IgG, is at least about 18 μg/mL. In some embodiments, the concentration of the non-specific antibody, such as a generic IgG, is at least about 20 μg/mL. In some embodiments, the concentration of the non-specific antibody, such as a generic IgG, is at least about 0.5 mg/mL.

Because of the high sensitivity of the disclosed methods, only a small amount of the sample is needed to detect the presence or absence of at least one tau protein in the sample. Accordingly, in some embodiments, the biological sample, such as the rhinorrhea or otorrhea sample, has a volume of from about 10 microliters to about 500 microliters. In some embodiments, the biological sample, such as the rhinorrhea or otorrhea sample, has a volume of from about 20 microliters to about 400 microliters. In some embodiments, the biological sample, such as the rhinorrhea or otorrhea sample, has a volume of from about 30 microliters to about 350 microliters. In some embodiments, the biological sample, such as the rhinorrhea or otorrhea sample, a volume of from about 40 microliters to about 300 microliters. In some embodiments, the biological sample, such as the rhinorrhea or otorrhea sample, has a volume of from about 45 microliters to about 250 microliters. In some embodiments, the biological sample, such as the rhinorrhea or otorrhea sample, has a volume of from about 50 microliters to about 200 microliters. In some embodiments, the biological sample, such as the rhinorrhea or otorrhea sample, has a volume of from about 60 microliters to about 150 microliters. In some embodiments, the biological sample, such as the rhinorrhea or otorrhea sample, has a volume of about 10, about 20, about 30, about 40, about 50, about 60, about 70, about 80, about 90, about 100, about 150, about 200, about 250, about 300, about 350, about 400, about 450, or about 500 microliters.

The disclosed methods can be used to rapidly detect the presence or absence of at least one tau protein in a biological sample obtained from a subject, making it possible to conveniently administer and analyze the results of the diagnostic assay, for example, in the presence of the subject, at the site of an injury, and/or by a trained or untrained professional and to provide the subject immediate medical attention if needed. In some embodiments, therefore, the presence or absence of at least one tau protein in the biological sample, such as the rhinorrhea or otorrhea sample, of the subject is detected in about 30 seconds to about 45 minutes after the step of contacting the sample with the first and/or second antibody or adding the sample to a device as disclosed herein. In some embodiments, the presence or absence of at least one tau protein in the biological sample, such as the rhinorrhea or otorrhea sample, of the subject is detected in about 45 seconds to about 40 minutes after the step of contacting the sample with the first and/or second antibody or adding the sample to a device as disclosed herein. In some embodiments, the presence or absence of at least one tau protein in the biological sample, such as the rhinorrhea or otorrhea sample, of the subject is detected in about 1 minute to about 30 minutes after the step of contacting the sample with the first and/or second antibody or adding the sample to a device as disclosed herein. In some embodiments, the presence or absence of at least one tau protein in the biological sample, such as the rhinorrhea or otorrhea sample, of the subject is detected in about 1 minute to about 20 minutes after the step of contacting the sample with the first and/or second antibody or adding the sample to a device as disclosed herein. In some embodiments, the presence or absence of at least one tau protein in the biological sample, such as the rhinorrhea or otorrhea sample, of the subject is detected in about 2 minutes to about 15 minutes after the step of contacting the sample with the first and/or second antibody or adding the sample to a device as disclosed herein. In some embodiments, the presence or absence of at least one tau protein in the biological sample, such as the rhinorrhea or otorrhea sample, of the subject is detected in about 30 seconds, about 45 seconds, about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 35 minutes, about 40 minutes, or about 45 minutes after the step of contacting the sample with the first and/or second antibody or adding the sample to a device as disclosed herein.

While the disclosed methods can be conducted by a trained professional in a hospital or laboratory setting, the disclosed methods and devices can be adapted for use by any person in a non-hospital or non-laboratory setting. Thus, in some embodiments, the disclosed methods are not conducted in a hospital setting. In some embodiments, the disclosed methods are not conducted in a laboratory setting. In some embodiments, the disclosed methods are conducted as a point-of-care diagnostic test.

Also provided herein, in some embodiments, is a method of treating a subject diagnosed with or suspected of having a TBI, said method comprising diagnosing the subject as having a TBI by any of the methods disclosed herein, and administering to the subject a therapeutically effective amount of treatment for the TBI. Depending on the severity of the TBI, the treatment can range from resting to seeking immediate emergency care. In some embodiments, the treatment for the TBI is resting. In some embodiments, the treatment for the TBI is administering to the subject a therapeutically effective amount of a pain reliever, an anti-seizure drug, a coma-inducing drug, and/or a diuretic. In some embodiments, the treatment for the TBI is surgery. In some embodiments, the treatment for the TBI is rehabilitation.

### Devices and Kits for Detection of CSF Leakage

Any of the methods described herein can be accomplished using a device that is adapted to carry out the method of detecting a tau protein in an appropriate biological sample using antibodies that bind to tau protein. In some embodiments, the device comprises a first anti-tau antibody that binds to the epitope of SEQ ID NO: 1 as described elsewhere herein and a second anti-tau antibody that binds to the epitope of SEQ ID NO: 2 as described elsewhere herein. The devices and kits disclosed herein can have multiple shapes and forms. In certain embodiments, the device can produce rapid results, such as a point-of-care, strip test. Thus, the device and methods disclosed herein can eliminate the need for expensive and time-consuming immunofixation electrophoresis tests, laboratory tests and radiologic tests.

In some embodiments, the devices and methods disclosed herein are easily adaptable for rapid, convenient use such that they can be used by an individual without medical training and/or prior experience. Of course, the devices and methods disclosed herein can also be used medical professionals in a hospital or other health care setting. In some embodiments, the device requires no special timing, dilutions, or concentrations prior to use. The device can detect low and high concentrations of a tau protein in a test sample. The simplicity of use and rapid results make the devices appropriate for use in surgery or in outpatient treatment, at home by a patient, or in any other setting. In some embodiments, therefore, the device is a point-of-care diagnostic device. In other embodiments, the device of the disclosure is an over-the-counter diagnostic device.

The device and methods disclosed herein may be designed to give a simple binary (e.g., yes/no) determination of the presence or absence of a tau protein in a test sample. A binary (e.g., yes/no) determination can be detected by a color change or other physical change. In some embodiments, the binary (e.g., yes/no) determination is a color change that is visible to human eyes. In other embodiments, the binary (e.g., yes/no) determination is a color change that is detected by a lateral flow reader. In some embodiments, the binary (e.g., yes/no) determination can be obtained in about 30 seconds to about 45 minutes after the step of contacting the sample with the first and/or second antibody or adding the sample to a device as disclosed herein. In some embodiments, the binary (e.g., yes/no) determination can be obtained in about 45 seconds to about 40 minutes after the step of contacting the sample with the first and/or second antibody or adding the sample to a device as disclosed herein. In some embodiments, the binary (e.g., yes/no) determination can be obtained in about 1 minute to about 30 minutes after the step of contacting the sample with the first and/or second antibody or adding the sample to a device as disclosed herein. In some embodiments, the binary (e.g., yes/no) determination can be obtained in about 1 minute to about 20 minutes after the step of contacting the sample with the first and/or second antibody or adding the sample to a device as disclosed herein. In some embodiments, the binary (e.g., yes/no) determination can be obtained in about 2 minutes to about 15 minutes after the step of contacting the sample with the first and/or second antibody or adding the sample to a device as disclosed herein. In some embodiments, the binary (e.g., yes/no) determination can be obtained in about 30 seconds, about 45 seconds, about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 35 minutes, about 40 minutes, or about 45 minutes after the step of contacting the sample with the first and/or second antibody or adding the sample to a device as disclosed herein. In some embodiments, the binary (e.g., yes/no) determination can be obtained in less than about 20 minutes, less than about 10 minutes, less than about 5 minutes, or less than about 1 minute after the step of contacting the sample with the first and/or second antibody or adding the sample to a device as disclosed herein.

The device disclosed herein may be configured in any manner suitable for providing a test area. In some embodiments, the device disclosed herein comprises a lateral flow strip test, also known as lateral flow immunoassay or immunochromatographic assay, or simply a strip test. Strip test technology offers a range of benefits including being user-friendly, relatively inexpensive and providing rapid results. A lateral flow test strip is composed of two main areas: a first antibody area (also referred to as non-immobilized antibody area or conjugate release area) and a test area (also referred to as a second antibody area). In some embodiments, the device disclosed herein is in a form of a test strip. In some embodiments, the device is in a form of a dipstick. In some embodiments, the device is in a form of a flow through device. In some embodiments, the device is in a form of a microfluidic device.

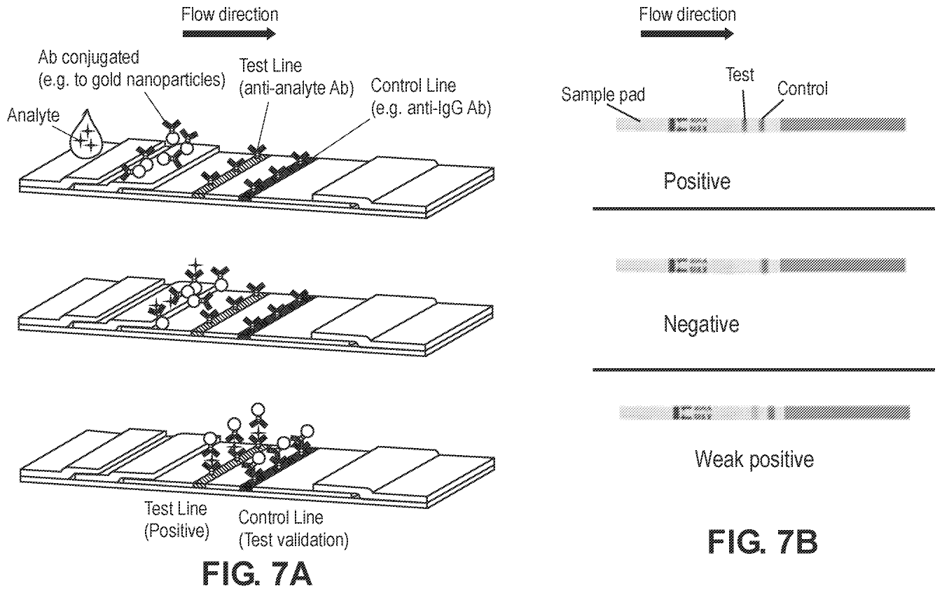

FIG. 5 illustrates a general lateral flow immunoassay device in the form of a test strip according to one embodiment of the disclosure. In essence, the test strip in FIG. 5 comprises a particle conjugate in the non-immobilized antibody area (or conjugate release area) and a nitrocellulose membrane (a lateral flow membrane) downstream of and configured for fluid communication with the non-immobilized antibody area. The non-immobilized antibody area (or conjugate release area) comprises a first anti-tau antibody that binds to a first epitope of the tau protein and the lateral flow membrane comprises a second anti-tau antibody immobilized thereon that binds to a second epitope of the tau protein. The test strip in FIG. 5 can optionally comprise a sample loading pad in the sample loading area upstream of and configured for fluid communication with the non-immobilized antibody area and/or an absorbent area (e.g., wick) downstream of and configured for fluid communication with the lateral flow membrane.

A sample loading pad in the sample loading area can comprise any material that allows for a flow-through of proteins and/or other molecules to be tested while filtering out any large particulate matter in a sample. A sample loading pad also functions to hold the sample so that it can slowly wick through into the non-immobilized antibody area (or conjugate release area) without overloading the test strip. There are two main types of materials that are commonly used as sample loading pads: cellulose fibers and woven meshes. In some embodiments, therefore, the sample loading pad comprises cellulose fibers. In other embodiments, the sample loading pad comprised in the disclosed devices comprises woven meshes. Any other material that is compatible with the design of the lateral flow device can be used in the sample loading area.

Once a test sample (or portion thereof) has migrated through the sample loading pad, it arrives at the non-immobilized antibody area (or conjugate release area) situated between the sample loading area and the lateral flow membrane. In some embodiments, the non-immobilized antibody area (or conjugate release area) comprises a non-immobilized antibody pad which contains a detector antibody conjugated to a detectable reagent. A detector antibody can be any antibody that specifically binds to a tau protein. Preferably, the detector antibody is the anti-tau antibody that binds to the epitope of SEQ ID NO: 2 as described herein. However, in certain embodiments, it is also possible to use the anti-tau antibody that binds to the epitope of SEQ ID NO: 1 as described herein as the detector antibody. A detector antibody is preferably labeled with a substance, such as a detectable reagent that can be visualized with the naked eye or with the aid of a machine as described herein. In some embodiments, the detector antibody is conjugated to an enzyme. In some embodiments, the detector antibody is conjugated to a colored latex particle. In other embodiments, the detector antibody is conjugated a gold nanoparticle. In some embodiments, the detector antibody is conjugated to a gold nano-shell.

The non-immobilized antibody pad can perform multiple tasks, including the uniform transfer of the detector reagent and test sample onto the membrane. When sample flows into the non-immobilized antibody pad, the detector reagent solubilizes, lifts off the pad material, and moves with the sample front into the membrane. The ideal non-immobilized antibody pad should have low non-specific binding, consistent flow characteristics, consistent bed volume, low extractables, good web handling characteristics, and consistent compressibility. In some embodiments, the non-immobilized antibody pad comprises glass fibers. In some embodiments, the non-immobilized antibody pad comprises cellulose fibers. In some embodiments, the non-immobilized antibody pad comprises surface-treated polyester filters. In some embodiments, the non-immobilized antibody pad comprises surface-treated polypropylene filters.

From the non-immobilized antibody pad, a sample (or portion thereof) migrates to the lateral flow membrane. The sample enters the lateral flow membrane and moves towards the distal end (i.e., towards the adsorbent area) of the test strip via capillary action. A lateral flow membrane can comprise any substance that allows for the flow-through of molecules especially proteins and antibodies. In some embodiments, the lateral flow membrane is a nitrocellulose membrane.

The lateral flow membrane comprises a capture antibody immobilized at a first location, sometimes called a test line. The capture antibody can specifically bind the antigen (e.g., tau protein) or the antigen-detector antibody complex (e.g., tau protein-detector antibody). By binding to the antigen or antigen-detector antibody complex, the capture antibody triggers a change in appearance in the first location (or test line), which can be visualized and, in some embodiments, quantified, for example, by a change in pattern or color. Preferably, the detector antibody is the anti-tau antibody that binds to the epitope of SEQ ID NO: 2 as described elsewhere herein, and the capture antibody is the anti-tau antibody that binds to the epitope of SEQ ID NO: 1 as described elsewhere herein. However, in certain other embodiments, the detector antibody may be the anti-tau antibody that binds to the epitope of SEQ ID NO: 1 as described elsewhere herein, and the capture antibody may be the anti-tau antibody that binds to the epitope of SEQ ID NO: 2 as described elsewhere herein. The presence of a pattern or color at the first location (or test line) is an indication of the presence of a tau protein in the test sample. The absence of such a pattern or color is an indication of a lack of a tau protein in the test sample.

In some embodiments, the lateral flow membrane further comprises a secondary antibody immobilized at a second location, sometimes called a control line, which is downstream of the first location. The secondary antibody serves as a control to show that the labeled (conjugated) detector antibody has moved from the non-immobilized antibody area to the second location on the lateral flow membrane via lateral flow. In certain embodiments, the secondary antibody is an anti-immunoglobulin antibody that specifically binds to the detector antibody regardless of whether an antigen is present. In some embodiments, the secondary antibody is a species-specific anti-IgG antibody (e.g., anti-mouse IgG antibody). By binding to the detector antibody, the secondary antibody triggers a change in appearance in the second location (or control line), which can be visualized and, in some embodiments, quantified, for example, by a change in pattern or color. The control line can also be used to provide a qualitative indication of the relative concentration of the tau protein in a test sample, for example, by using a detectable reporters and a calibration curve to calibrate the amount of such protein.

The absorbent area, typically at the distal end of the test strip, is downstream of and configured for fluid communication with the lateral flow membrane. In some embodiments, the absorbent area comprises an absorbent pad which serves as a reservoir to hold the sample after it has wicked across the lateral flow membrane for a short period of time before the sample begins to flow back across the membrane towards the proximal end. In some embodiments, the absorbent pad comprises cellulose fibers. In other embodiments, the absorbent pad comprises woven meshes. Any other material that is compatible with the design of the lateral flow device can be used in the absorbent area.

The test strip in FIG. 5 can optionally comprises a solid substrate as a backing to hold all the components of the test strip in place. The test strip may be sized and shaped to be received within a housing. The housing can be formed of injection molded plastic, or any other suitable material. Thus, in some embodiments, the test strip is located inside a housing or other solid support, such as, for example, a plastic housing. In such embodiments, the housing can have a hole located over the sample loading pad. The housing can also have windows covering the test line(s) and control line(s). In some embodiments, a section of the sample loading pad can be seen through the hole and a section of the test line(s) and control line(s) can be seen through their respective windows. In some embodiments, only one window covering the test line(s) and control line(s).

In a variation of the test strip described above, the control line is composed of immobilized antigen (e.g., a tau protein). The introduction of a sample to the test strip results in the migration of at least some unbound detector antibody from the non-immobilized antibody pad to the control line where the conjugated detector antibody binds the immobilized antigen and creates a visual appearance (e.g., a color change).

In another variation of the embodiments described above, the device includes a first control line and a second control line distal to the first control line. The first control line contains a detector antibody. This detector antibody does not necessarily have to bind to the antigen of interest (e.g., a tau protein) but must be conjugated to a detectable reagent. The second control line contains an immobilized capture antibody that specifically binds the detector antibody of the first control line. The introduction of a fluid sample to the test strip results in the migration of the detector antibody from the first control line towards the absorbent pad resulting in a formation of a first control detector antibody-second control capture antibody complex, which can be visually detected.

In yet another variation of the embodiments described herein, the non-immobilized antibody pad comprises more than one detector antibody conjugated to a detectable reagent. In some embodiments, each detector antibody binds to a different epitope of the tau protein. In some embodiments, the antibodies are monoclonal antibodies. A test strip having more than one detector antibody can optionally have more than one test line. In some embodiments, multiple test lines are adjacent to one another either laterally or transversely in the lateral flow membrane.

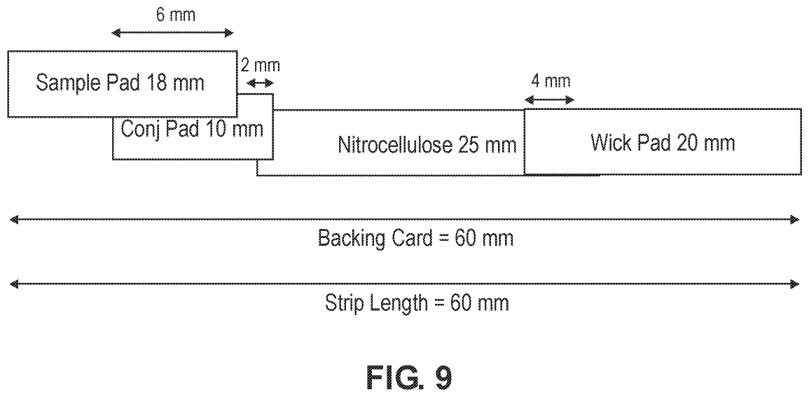

The devices of the disclosure can be configured in any suitable sizes. An exemplary configuration of a device according to one embodiment is provided in FIG. 9. In this configuration, the device is in form of a test strip with a length of about 60 mm and comprises a sample loading pad of about 18 mm in length, a conjugate pad of about 10 mm in length, a nitrocellulose membrane of about 25 mm in length, and a wick pad of about 20 mm in length. The positioning of these components is such that the sample loading pad is located on one end of the test strip and overlaps with the conjugate pad by about 6 mm in length, the wick pad is located on the other end of the test strip and overlaps with the nitrocellulose membrane by about 4 mm in length, and the conjugate pad and the nitrocellulose membrane overlap with each other at the free end by about 2 mm in length. All the components are held in place by a backing card.

The devices of the disclosure are highly sensitive to tau protein and can produce results with high degree of accuracy. In some embodiments, the device has a sensitivity of detecting the tau protein in a sample at a concentration of from about 0.1 pg/μl to about 10 pg/μl. In some embodiments, the device has a sensitivity of detecting the tau protein in a sample at a concentration of from about 0.1 pg/μl to about 5 pg/μl. In some embodiments, the device has a sensitivity of detecting the tau protein in a sample at a concentration of from about 0.1 pg/μl to about 4 pg/μl. In some embodiments, the device has a sensitivity of detecting the tau protein in a sample and at a concentration of from about 0.1 pg/μl to about 3 pg/μl. In some embodiments, the device has a sensitivity of detecting the tau protein in a sample at a concentration of from about 0.1 pg/μl to about 2 pg/μl. In some embodiments, the device has a sensitivity of detecting the tau protein in a sample at a concentration of from about 0.1 pg/μl to about 1 pg/μl. In some embodiments, the device has a sensitivity of detecting the tau protein in a sample at a concentration of from about 0.2 pg/μl to about 1 pg/μl. In some embodiments, the device has a sensitivity of detecting the tau protein in a sample at a concentration of from about 0.3 pg/μl to about 1 pg/μl. In some embodiments, the device has a sensitivity of detecting the tau protein in a sample at a concentration of from about 0.5 pg/μl to about 1 pg/μl. In some embodiments, the device has a sensitivity of detecting the tau protein in a sample at a concentration of from about 0.5 pg/μl to about 0.8 pg/μl.

In some embodiments, the devices disclosed herein may further comprise a non-specific antibody as described herein to reduce or eliminate any potential non-specific binding between the sample and the first and/or second anti-tau antibodies. In some embodiments, the non-specific antibody is added in the sample loading pad. In some embodiments, the non-specific antibody is added in the non-immobilized antibody area. In some embodiments, the non-specific antibody is added in the lateral flow membrane. In some embodiments, the non-specific antibody is added in the first location on the lateral flow membrane. In some embodiments, the non-specific antibody is added in the non-immobilized antibody area and the first location on the lateral flow membrane. In some embodiments, the non-specific antibody is added in the non-immobilized antibody area and the first location on the lateral flow membrane by adding the non-specific antibody into the storage buffer for the first and/or second anti-tau antibodies.

Any non-specific antibody from any species may be used. In some embodiments, the non-specific antibody is an IgG antibody. In some embodiments, the IgG antibody is a non-specific mouse IgG.

When in use, the concentration of the non-specific antibody should be sufficient to block any potential non-specific binding between the sample and the first and/or second anti-tau antibodies. In some embodiments, the concentration of the non-specific antibody used in the devices disclosed herein is from about 0.1 mg/mL to about 5.0 mg/mL. In some embodiments, the concentration of the non-specific antibody is from about 0.5 mg/mL to about 4.5 mg/mL. In some embodiments, the concentration of the non-specific antibody is from about 0.5 mg/mL to about 4.0 mg/mL. In some embodiments, the concentration of the non-specific antibody is from about 0.5 mg/mL to about 3.5 mg/mL. In some embodiments, the concentration of the non-specific antibody is from about 0.5 mg/mL to about 3.0 mg/mL. In some embodiments, the concentration of the non-specific antibody is from about 0.5 mg/mL to about 2.5 mg/mL. In some embodiments, the concentration of the non-specific antibody is from about 0.5 mg/mL to about 2.0 mg/mL. In some embodiments, the concentration of the non-specific antibody is from about 0.5 mg/mL to about 1.5 mg/mL. In some embodiments, the concentration of the non-specific antibody is at least about 0.5, 0.6, 0.7, 0.8, 0.9 or 1.0 mg/mL. In some embodiments, the concentration of the non-specific antibody is at least about 0.5 mg/mL.

In addition to or as an alternative to using a non-specific antibody, one or more different non-specific blocking agents may be used. The additional non-specific blocking agents, include, but are not limited to commercial blocking agents (STABILGUARD™ STABILBLOCK™, STABILCOAT™; Surmodics, Eden Prairie, Minnesota), fetal calf or bovine serum, heat-inactivated serum (typically from the same species as the antibodies being used in the detection assay), bovine serum albumin, casein protein, non-fat milk, or gelatin. In one embodiment, the additional non-specific blocking agent is added to the sample loading pad before the sample is loaded onto the pad.

Because of the high sensitivity of the disclosed devices, only a small amount of the sample is needed to detect the presence or absence of the tau protein in the sample. Accordingly, in some embodiments, the sample loading pad is configured to receive a fluidic sample of about 10 microliters to about 500 microliters. In some embodiments, the sample loading pad is configured to receive a fluidic sample of from about 20 microliters to about 400 microliters. In some embodiments, the sample loading pad is configured to receive a fluidic sample of from about 30 microliters to about 350 microliters. In some embodiments, the sample loading pad is configured to receive a fluidic sample of from about 40 microliters to about 300 microliters. In some embodiments, the sample loading pad is configured to receive a fluidic sample of from about 45 microliters to about 250 microliters. In some embodiments, the sample loading pad is configured to receive a fluidic sample of from about 50 microliters to about 200 microliters. In some embodiments, the sample loading pad is configured to receive a fluidic sample of from about 60 microliters to about 150 microliters. In some embodiments, the sample loading pad is configured to receive a fluidic sample of about 10, about 20, about 30, about 40, about 50, about 60, about 70, about 80, about 90, about 100, about 150, about 200, about 250, about 300, about 350, about 400, about 450, or about 500 microliters.

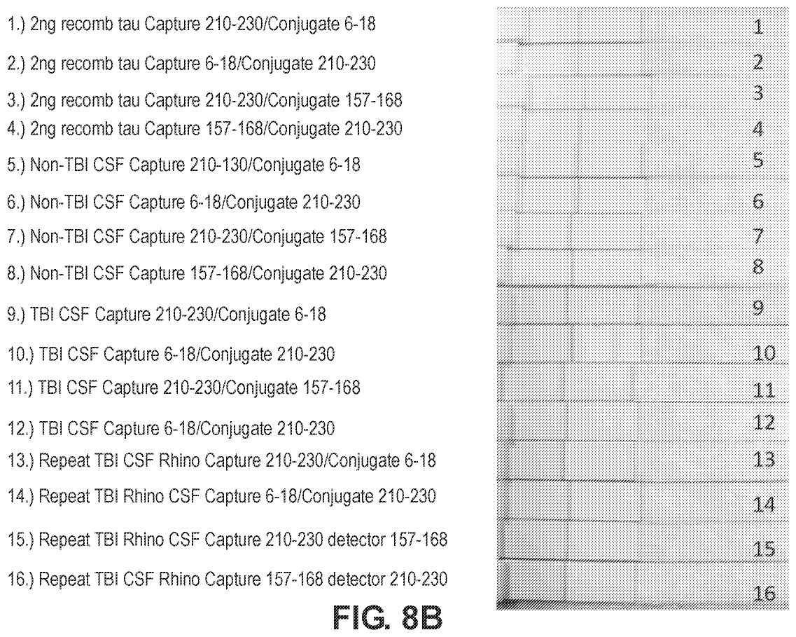

When reading a test assay using the devices disclosed herein, even the faintest visible change should be considered a result. There are four possible outcomes that can be observed using the devices disclosed herein having a test line and a control line. A first possible outcome is that two lines appear, one in the test line and one in the control line. This indicates a positive assay and indicates that the subject has a CSF leakage and may suffer from conditions associated therewith. The second possible outcome is a single line in the control line. This may be a valid negative result. A third possible outcome is a positive test line but no control line. This indicates a faulty assay and requires rerunning the assay on a new strip test. A fourth possible outcome is that no lines appear. This may also be the result of a faulty assay and a new assay should be conducted. Illustrative positive and negative results are shown in FIG. 7B

Also provided herein are kits comprising any of the devices disclosed herein. Such kits can be used for detection of CSF leakage in a subject. Kits may include materials and reagents adapted to selectively detect the presence of a tau protein in a sample obtained from a subject. In some embodiments, the kits further comprise an object to facilitate collection of a biological sample, such as a rhinorrhea or otorrhea sample. In some embodiments, the object for collecting the sample is a swab comprising an absorbent material configured to absorb cerebrospinal fluids. In some embodiments, the kits further comprise one or more reagents, such as dilution buffers and wash buffers. In some embodiments, the one or more reagents contain PBS, Triton X-100, and sodium azide. The PBS serves to adjust the sample to a neutral pH of 7 such that the antibodies will be able to function properly. The Triton X-100 is a surfactant that helps prevent aggregates from forming and blocking the flow across the lateral flow membrane. Sodium azide is a general preservative and disinfectant which helps to prevent growth of any microbial contaminants during storage of the buffer. Other optional buffers and solutions may also be added to the kits as necessary.

In some embodiments, the kits of the disclosure can include components that are useful in the procedures herein including, but not limited to, capture reagents, developing reagents, reacting surfaces, substrates, means for detection of control samples, instructions and interpretive information explaining how to read results. In some embodiments, the kits contain instructions, such as directions conveying any one or more of the method steps disclosed herein.

The disclosed devices and kits can be used to practicing any of the methods disclosed herein elsewhere. Accordingly, in some embodiments, the disclosed devices and kits are used to diagnose CSF leakage by detecting the presence of a tau protein in sample obtained from a subject, such as tears, saliva, nasal discharge, ear discharge, or any other tissue or bodily fluid aside from CSF and perilymph. As CSF leakage is associated with various conditions including, but not limited to, rhinorrhea, otorrhea, recurrent meningitis, chronic headaches, neck aches, loss of hearing (see Patel et al., Ear Nose Throat. J., 2000, 79 (5): 372-3, 376-8), the disclosure also provides for a diagnosis of conditions associated with CSF leakage.

Also provided herein, in some embodiments, is a method of diagnosing a head trauma, spine injury, skull base defects, high pressure intracranial hydrocephalus, or intracranial hypertension in a subject in need thereof, the method comprising detecting the presence or absence of at least one tau protein in a biological sample from the subject, the method comprising detecting the presence or absence of at least one tau protein in a biological sample from the subject using any of the devices disclosed herein or a kit comprising the same. In some embodiments, the head trauma is a TBI. In some embodiments, the TBI is a concussion.

Also provided is a method of monitoring progression of recovery from a head trauma, spine injury, skull base defects, high pressure intracranial hydrocephalus, or intracranial hypertension in a subject in need thereof, the method comprising detecting the presence or absence of at least one tau protein in a biological sample from the subject using any of the devices disclosed herein or a kit comprising the same. In some embodiments, the head trauma is a TBI. In some embodiments, the TBI is a concussion.

Further provided is a method of treating a subject diagnosed with or suspected of having a head trauma, spine injury, skull base defects, high pressure intracranial hydrocephalus, or intracranial hypertension, the method comprising: a) detecting the presence or absence of at least one tau protein in a biological sample from the subject using any of the devices disclosed herein or a kit comprising the same; and b) administering to the subject a therapeutically effective amount of treatment for the head trauma, spine injury, skull base defects, high pressure intracranial hydrocephalus, or intracranial hypertension if the presence of the at least one tau protein is detected. In some embodiments, the head trauma is a TBI and the treatment comprises one or more of: a) resting; b) administering to the subject a therapeutically effective amount of a pain reliever, an anti-seizure drug, a coma-inducing drug, and/or a diuretic; c) surgery; and d) rehabilitation. In some embodiments, the TBI is a concussion.

In any of these methods, the biological sample may be obtained from a nose or an ear of the subject. In some embodiments, the biological sample is obtained by swabbing the nose of the subject using a swab. In some embodiments, the biological sample is obtained by swabbing the ear of the subject using a swab. In some embodiments, the detection of the presence or absence of at least one tau protein in a biological sample from the subject using any of the devices disclosed herein or a kit comprising the same is not conducted in a hospital or laboratory setting.

In some embodiments, detection of the tau protein is made by adding the test sample to the device disclosed herein, e.g., by contacting a test sample with the sample loading pad of the disclosed device. A test sample can comprise any fluid, including but not limited to, tears, saliva, nasal discharge, ear discharge, or any other bodily fluid aside from CSF and perilymph. A test sample may also comprise tissue or cells. The sample does not need to be diluted or concentrated before applying it to the sample loading pad, but it can be diluted or concentrated before applying to the sample loading pad if the situation requires. In some embodiments, a tissue sample or fluid can be directly contacted with sample loading pad. This is especially useful in surgery or when there is nose or ear discharge. In some embodiments, a test sample is diluted before applying it to the sample loading pad. In some embodiments, a test sample is concentrated before applying it to the sample loading pad. In other embodiments, a test sample is obtained from a subject and is applied to a sample loading pad inside a window of a housing using a pipette.

As the methods and devices disclosed herein are easy to use, any person including a patient can perform the immunoassays. Furthermore, the ease of use allows these assays to be performed at any location including, for example, at home, at a sporting event or other indoor or outdoor activity, at a place of employment, in military combat or training, in a hospital or clinic. For example, the immunoassays disclosed herein can be performed immediately after a trauma or a head injury. They can also be performed during-surgery or post-surgery, especially in head and brain surgery. Also contemplated herein is the repetitive use of the disclosed methods and immunoassays to detect its onset or recurrence, especially in testing individuals previously diagnosed with CSF leakage, those who are suspected of having CSF leakage, and those who are at risk of developing CSF leakage. Individuals suspected of having CSF leakage include those having recently experienced trauma or showing symptoms such as headaches, nose aches, or loss of hearing. Such individuals can be tested (or perform self-evaluation) using the devices and methods disclosed herein daily, weekly, monthly, quarterly, or bi-annually. A self-evaluation can be as simple as placing a sample on the sample loading pad or placing the sample loading pad inside a nostril and observing a color change (or lack thereof) in the test line. It is further contemplated that the detection methods and devices disclosed herein can be used to monitor CSF leakage to evaluate treatment options for a patient. Treatments for CSF leakage include, for example, CSF diversion through lumbar drain and primary surgical repair.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1. Epitope Mapping of Tau in CSF

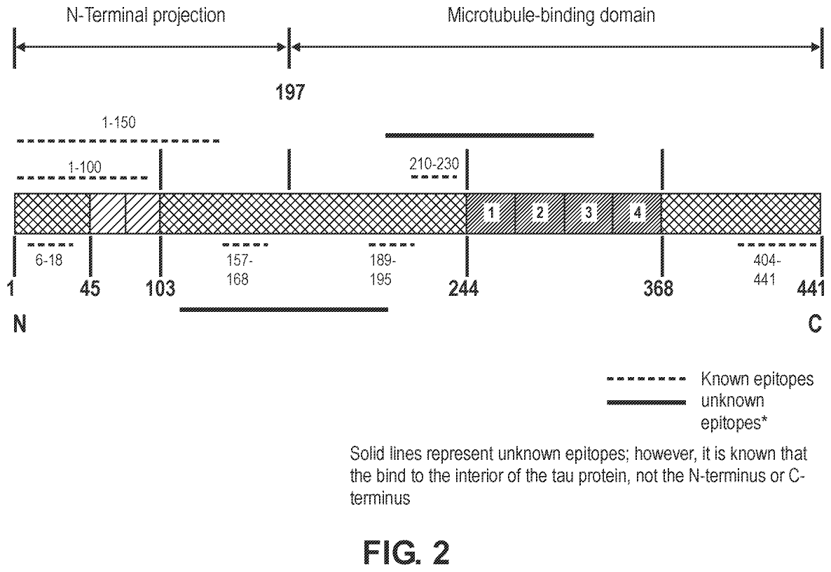

To develop a definitive diagnostic for the detection of tau protein in CSF, it is necessary to first determine which tau antibody binding sites (epitopes) were predominantly available in the proteolytically modified tau protein in CSF. To this end, different antibodies that bound different regions of the tau protein shown in FIG. 2 were selected and tested. These include anti-tau antibodies that bind to the epitopes 6-18, 1-100, 1-150, 157-168, 189-195, 210-230, 404-441, and two anti-tau antibodies that bind to the interior of the tau protein with their corresponding epitope sequence unknown.

As the concentration of the full-length tau protein, differentially spliced tau protein isoforms, and proteolytic fragments thereof in CSF varies greatly from person to person, ranging from approximately 1 μg to 5 ng (internal data not published), it is important to find an antibody that binds to as many tau proteolytic fragments and tau protein isoforms as possible to ensure a strong detection signal in a diagnostic assay. Antibodies that do not bind tau proteolytic fragments and/or tau protein isoforms found in CSF or bind to only a limited number of tau fragment or tau fragments that are present in low concentrations will generate little or no detectable signal and thus would not be effective in a diagnostic assay.

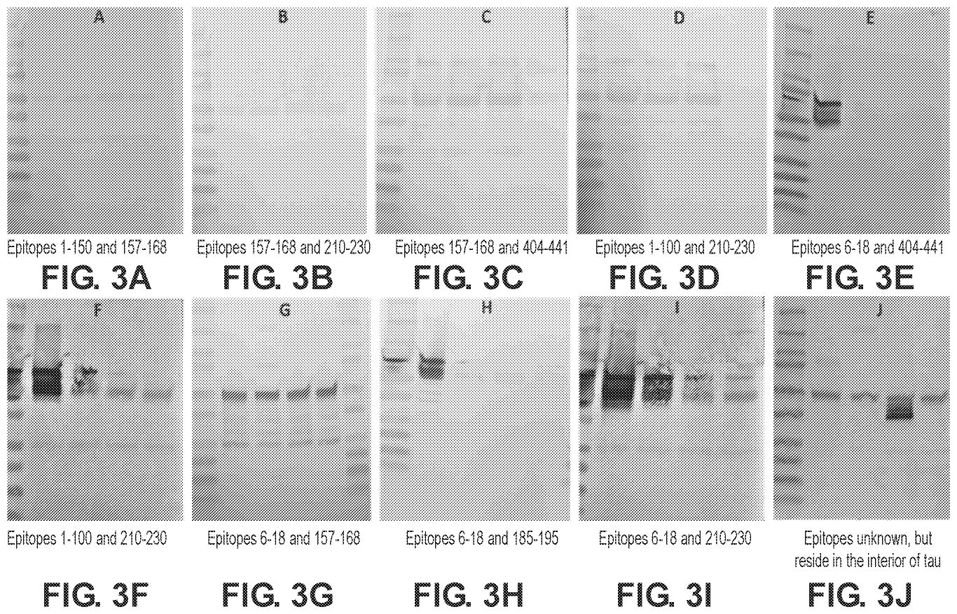

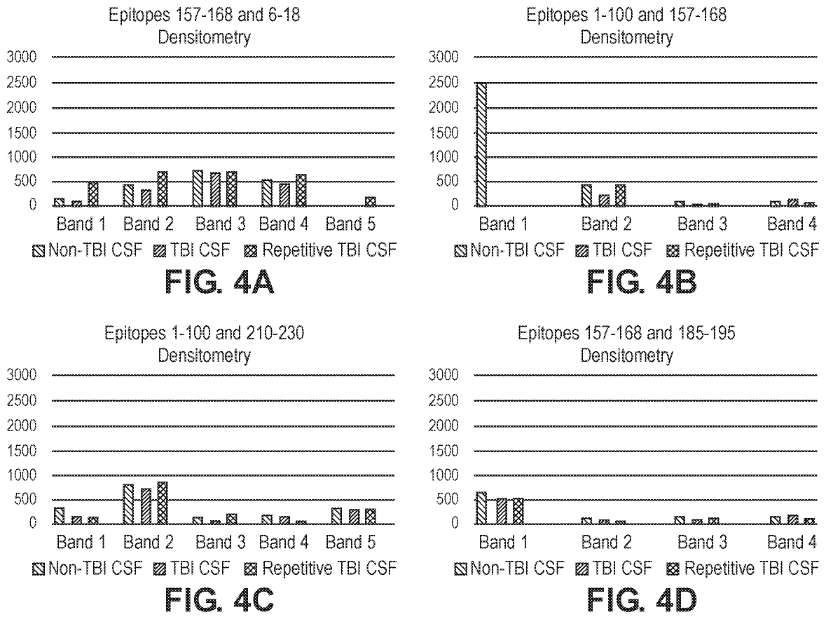

A series of immunoprecipitation reactions using various pairs of antibodies outlined in FIG. 2 were performed to determine which antibodies could be used to effectively detect tau protein for diagnostic purposes in CSF. In this screening, a first anti-tau antibody was immobilized on protein A Sepharose and the tau proteins were immunoprecipitated from various human donor CSF samples (both from TBI and non-TBI). The reaction supernatant was then removed, the protein A Sepharose was washed, the immunoprecipitated tau proteins were run on SDS-PAGE (sodium dodecyl sulfate-polyacrylamide gel electrophoresis) and probed with a second anti-tau antibody that recognizes an epitope on tau that is different than the tau epitope recognized by the immobilized, first anti-tau antibody. The results of these experiments are shown in FIG. 3A-3J.

Each of FIG. 3A-3J consists of 5 lanes for the Western blot analysis. Lane 1 is the molecular weight standards which represent the molecular weights of 250 kDa, 150 kDa, 100 kDa, 75 kDa, 50 kDa, 37 kDa, 25 kDa, 20 kDa, 15 kDa, and 10 kDa, from top to bottom. Lane 2 contains purified recombinant tau protein as a positive control and lanes 3-5 each contains different pooled donor CSF samples. In FIG. 3A, the anti-tau antibody that binds to epitope 1-150 was immobilized for the immunoprecipitation and the anti-tau antibody that binds to epitope 157-168 was used to probe the Western blot. In FIG. 3B, the anti-tau antibody that binds to epitope 157-168 was immobilized for the immunoprecipitation and the anti-tau antibody that binds to epitope 210-230 was used to probe the Western blot. In FIG. 3C, the anti-tau antibody that binds to epitope 157-168 was immobilized for the immunoprecipitation and the anti-tau antibody that binds to epitope 404-441 was used to probe the Western blot. In FIG. 3D, the anti-tau antibody that binds to epitope 1-100 was immobilized for the immunoprecipitation and the anti-tau antibody that binds to epitope 210-230 was used to probe the Western blot. In FIG. 3E, the anti-tau antibody that binds to epitope 6-18 was immobilized for the immunoprecipitation and the anti-tau antibody that binds to epitope 404-441 was used to probe the Western blot. In FIG. 3F, the anti-tau antibody that binds to epitope 1-100 was immobilized for the immunoprecipitation and the anti-tau antibody that binds to epitope 210-230 was used to probe the Western blot. In FIG. 3G, the anti-tau antibody that bind to epitope 6-18 was immobilized for the immunoprecipitation and the anti-tau antibody that binds to epitope 157-168 was used to probe the Western blot. In FIG. 3H, the anti-tau antibody that binds to the epitope 6-18 was immobilized for the immunoprecipitation and the anti-tau antibody that binds to epitope 185-195 was used to probe the Western blot. In FIG. 3I, the anti-tau antibody that binds to epitope 6-18 was immobilized for the immunoprecipitation and the anti-tau antibody that binds to the epitope 210-230 was used to probe the Western blot. In FIG. 3J, the two anti-tau antibodies bind to the interior of the tau protein but the specific epitope sequence that is recognized by each antibody is unknown.

As shown in FIG. 3G, the pair of anti-tau antibodies that bind to epitopes 6-18 and 157-168 recognize the most tau proteolytic fragments and/or tau protein isoforms as demonstrated by the protein banding patterns observed in the Western blot with the following molecular weights: 75 kDa, 50 kDa, 35 kDa, 30 kDa, 25 kDa, and 17 kDa. Although some protein bands detected have molecular weights higher than the full-length tau protein, this may be explained by post-translational modifications, because it is known that the tau protein is heavily modified post-translationally. Two unique protein bands of molecular weights 17 kDa and 30 kDa were observed with this particular pair of anti-tau antibodies, which were not observed with other pairs of anti-tau antibodies tested.

### Example 2. Semi-Quantitative Analysis of Anti-Tau Antibodies Binding Affinity

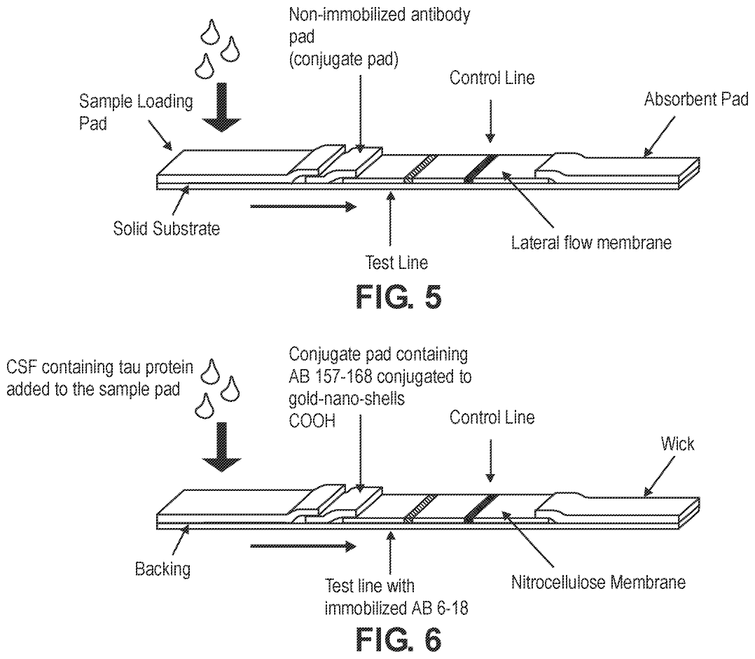

Another important aspect in maximizing the signal intensity of an immunoassay is the binding affinity of the antibodies. To this end, a semi-quantitative analysis using a densitometry software (Image Studio Lite Ver. 5.2) was performed to determine the binding affinity of each of the anti-tau antibody combinations. The results of this semi-quantitative analysis are shown in FIG. 4A-FIG. 4D.

The sample types shown in each of FIG. 4A-FIG. 4D are: TBI CSF sample (lumbar CSF) shown in yellow, and repetitive TBI CSF sample (rhinorrhea CSF) shown in green. FIG. 4A shows the densitometry results for 5 of the tau proteolytic fragments observed on the Western blot analysis for the antibody pair recognizing epitopes 6-18 and 157-168. FIG. 4B shown the densitometry results for the 4 tau proteolytic fragments observed on the Western blot analysis for the antibody pair recognizing epitopes 1-100 and 157-168 (the 5th band was not detected with this antibody pair). FIG. 4C shows the densitometry results for the 5 tau proteolytic fragments observed on the Western blot analysis for the antibody pair recognizing epitopes 1-100 and 210-230. FIG. 4D shows the densitometry results for the 4 tau proteolytic fragments observed on the Western blot analysis for the antibody pair recognizing epitopes 157-168 and 185-195 (the 5th band was not detected with this antibody pair).

The results in FIG. 4A-FIG. 4D demonstrate that, not only does the antibody pair that recognizes epitopes 6-18 and 157-168 detect the most tau proteolytic fragments, but they also consistently provide a more robust signal for most of the tau bands for the other two types of CSF samples (TBI lumbar CSF and repetitive TBI CSF rhinorrhea).

### Example 3. Lateral Flow Immune Assay with Anti-Tau Antibodies

To assess whether the antibody pair that recognizes epitopes 6-18 and 157-168 is suitable for use in a diagnostic platform, a lateral flow immunoassay was employed to compare this pair of antibodies with other antibody pairs that did not perform as well.

The principle behind a lateral flow immune assay is a liquid sample (e.g., CSF) containing the analyte of interest (e.g., tau protein) moves without the assistance of external forces (e.g., via capillary action) though a polymeric strip (typically nitrocellulose), on which specific antibodies that are attached can interact with the analyte of interest. A general lateral flow immunoassay design is depicted in FIG. 5, which can contain two overlapping membranes that are mounted on a backing material for better stability and handling. The design can include: (1) a sample loading pad where the sample containing the analyte of interest is applied; (2) a non-immobilized antibody pad, or conjugate pad, where the conjugated antibody has been stored; (3) a lateral flow membrane with immobilized antibodies where the analyte and bound conjugated antibody are captured to generate the test line; and (4) an absorbent pad which facilities the flow of the fluid through the device.

An example of a lateral flow immunoassay using the anti-tau antibodies recognizing epitopes 6-18 and 157-168 is shown in FIG. 6. In this exemplary design, the non-immobilized antibody pad, or conjugate pad, contains the antibody recognizing epitope 157-168 (AB 157-168) conjugated to gold nano-shells and the lateral flow membrane contains the antibody recognizing epitope 6-18 (AB 6-18) immobilized on the test line and an anti-IgG antibody immobilized on the control line.

A mechanism of action of such a lateral flow immunoassay is illustrated in FIG. 7A. As shown on the top of FIG. 7A, the sample containing tau proteins (“Analyte”) is deposited on the sample loading pad and migrates towards the conjugate pad. A first anti-tau antibody conjugated to a label (“conjugated Ab”) binds to the tau proteins, if present in the sample (middle of FIG. 7A), and migrates to the test line, where the bound tau proteins are captured with a second anti-tau antibody (“capture antibody”) that is immobilized to the lateral flow membrane. The conjugated antibody that is not captured at the test line will continue to flow toward the control line, which contains an immobilized secondary antibody specific for the conjugated antibody (e.g., species-specific anti-immunoglobulin). The capture of the conjugated antibody by the immobilized secondary antibody provides a positive control to show that the conjugated antibody has moved from the conjugate pad to the control line via lateral flow. An exemplary read out of such a lateral flow immunoassay is shown in FIG. 7B. If the sample contains the analyte, both test line and control line will appear and, depending on the amount of the analyte in the sample, the test line can appear as a strong signal (“Positive” in FIG. 7B) or a weak signal (“Weak positive” in FIG. 7B). Conversely, if there is no analyte in the sample, only the control line will appear (“Negative” in FIG. 7B).

Various combinations of anti-tau antibodies that recognize different epitopes of the tau protein were tested using this design, with a first antibody that binds to one tau epitope conjugated to 150 nm gold nano-shells and a second antibody that binds to a different tau epitope immobilized on the lateral flow membrane. The resultant lateral flow immunoassays were tested with various CSF samples to determine whether they can perform functionally in a diagnostic device-type setting. It was found that, among all the anti-tau antibodies tested, the anti-tau antibodies that bind to epitopes 6-18 and 157-168 (AB 6-18 and AB 157-168) perform the best with some exemplary results shown in FIG. 8A-8B. As shown in FIG. 8A, the anti-tau antibodies that recognized epitopes 6-18 and 157-168 (AB 6-18 and AB 157-168), when used in a lateral flow diagnostic platform, consistently generated a strong, positive signal as a visible line at the test line (Line 1), as well as a second visible line at the control line (line 2) for the recombinant tau protein (positive control; top three panels) and for the three different types of clinical samples, non-TBI CSF, TBI CSF, and repeat TBI CSF from rhinorrhea. Conversely, the anti-tau antibodies that recognize various other epitope combinations aside from epitopes 6-18 and 157-168, when used in a lateral flow diagnostic platform, produced only a visible line at the control line but did not result in a detectable signal in the test line as shown in FIG. 8B. The antibody combinations tested and shown in FIG. 8B include the antibodies that bind to epitopes 6-18 and 210-230 and the antibodies that bind to epitopes 157-168 and 210-230. Thus, even other antibodies combinations that included one of the antibodies that recognized either epitope 6-18 or epitope 157-168—but not both—failed to provide a detectable signal, demonstrating the specificity of the antibody pair required to achieve a detectable signal in this lateral flow diagnostic platform.

It was surprisingly found that the strong, positive signal generated by the anti-tau antibodies that bind to epitopes 6-18 and 157-168 (AB 6-18 and AB 157-168) in the lateral flow diagnostic platform described in Example 3 could only be consistently obtained when the antibody that binds to epitope 157-168 is the conjugated antibody that is labeled and located in the non-immobilized antibody area and the antibody that binds to epitope 6-18 is the capture antibody immobilized on the lateral flow membrane. When the anti-tau antibody that binds to epitope 6-18 was used as the conjugated antibody and the anti-tau antibody that binds to epitope 157-168 was used as the capture antibody, no visible signal could be detected. As shown in FIG. 8C, a clear, visible test line can only be seen in Lanes 1, 3, and 5 where the antibody that binds to epitopes 157-168 was used as the conjugated antibody. Conversely, when the antibody that binds to epitopes 6-18 was used as the conjugated antibody, no test line was shown (FIG. 8C, Lanes 2, 4, and 6). The control line appearing in each lane indicates that the conjugated antibody has properly migrated to the area where the secondary antibody is immobilized on the lateral flow membrane via lateral flow.

### Example 4. Lateral Flow Immunoassay Devices with Anti-Tau Antibodies AB 6-18 and AB 157-168

An exemplary lateral flow immunoassay device according to the disclosure may comprise the following components with the component positioning shown in FIG. 9.

| Condition | Exemplary Mode |
|---|---|
| Nitrocellulose membrane | MDI150 |
| Capture antibody | Anti-Tau, 6-18 antibody (clone |
| | Tau 12) @ 2 mg/ml |
| Conjugate nanoparticle | 150 nm gold nano-shells |
| Conjugation reaction buffer | 5 mM KPhos |
| Conjugate antibody | Anti-Tau, 157-168 antibody (clone |
| | 2G9.F10) @ 50 μg/ml with 0.5% BSA |
| Conjugate pad | Pretreated GFDX |
| Sample pad | Pretreated GFDX |
| Cassette | Sheng Feng |

An alternative exemplary lateral flow immunoassay device according to the disclosure may comprise the following components with the component positioning shown in FIG. 9

| Condition | Exemplary Mode |
|---|---|
| Nitrocellulose membrane | Vivid 120 |
| Capture antibody | Anti-Tau, 6-18 antibody @ 2 mg/mL |
| | in 10 mM acetate, pH 6.0 |
| Conjugate nanoparticle | 150 nm gold nano-shells |
| Conjugation reaction buffer | 2 mM NaPhos & 0.01X PBS |
| Conjugate antibody | Anti-Tau, 157-168 antibody @ 20 μg/mL |
| Storage buffer | 0.5X PBS, 0.5% Casein, 0.5% BSA, 1% |
| | Tween 20, 0.05% Sodium Azide, pH 8.0 |
| Conjugate pad | Pretreated GFDX |
| Sample pad | Pretreated GFDX (10 mM Tris, 250 mM |
| | NaCl, 2% BSA, 0.25% Tween 20, 20% |
| | Sucrose, 0.3% Sodium Deoxycholate, |
| | 18 μg/mL HBR-11*, pH 8.0) |
| Cassette | Sheng Feng |

*HBR-11, a heterophilic blocking reagent (HBR), is a generic murine antibody cocktail used as a blocking agent for non-specific interactions.

When in use, 100 μL of a sample (e.g., a rhinorrhea or otorrhea sample from a subject) is premixed with 100 μL of nasal wash and 400 μL of buffer (10 mM Tris, 0.05% sodium azide, pH 7.9) prior to loading onto the sample pad of the device.

### Example 5. Production of Anti-Tau Antibodies AB 6-18 and AB 157-168 in CHO Cells

The anti-tau antibody that specifically binds to the first epitope of SEQ ID NO: 1 (i.e., anti-tau antibody AB 6-18) and the anti-tau antibody that specifically binds to the second epitope of SEQ ID NO: 2 (i.e., anti-tau antibody AB 157-168) were recombinantly produced in animal cells. Briefly, the nucleic acids encoding the heavy and light chains were cloned into a vector system using conventional (non-PCR based) cloning techniques. The vector plasmids were gene synthesized. Plasmid DNA was prepared under low-endotoxin conditions based on anion exchange chromatography. DNA concentration was determined by measuring the absorption at a wavelength of 260 nm.

The recombinant antibodies were expressed in suspension-adapted CHO K1 cells (originally received from ATCC and adapted to serum-free growth in suspension culture). Following transfection of the plasmid DNA, cells were grown in an animal-component free, serum-free medium. Supernatant was harvested by centrifugation and subsequent filtration (0.2 μm filter). The antibody was then purified using MABSELECT SURE™ (Cytiva). In this way, recombinant anti-tau antibody AB 6-18 produced in CHO K1 cells (CHO 6-18) and a recombinant anti-tau antibody AB 157-168 produced in CHO K1 cells (CHO 157-168) were synthesized.

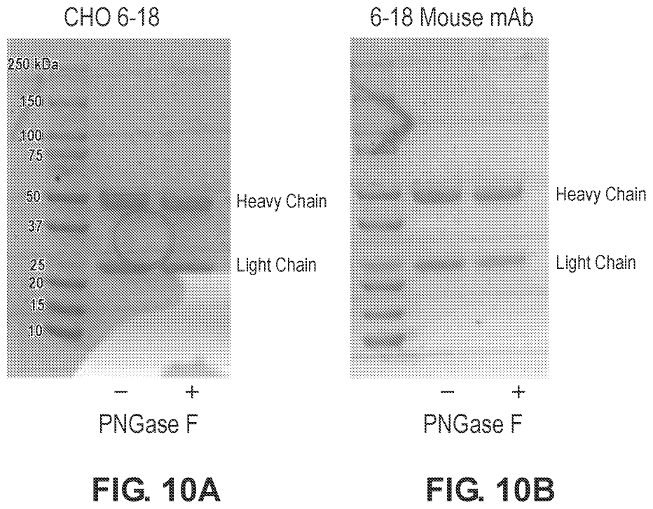

The CHO 6-18 antibody was compared to a mouse monoclonal antibody (6-18 mouse mAb) to compare any potential differences in glycosylation. The glycosylation pattern of a recombinant human protein, such as an antibody, is dependent upon the type of host cell or organism used to express the recombinant protein. The antibodies were either untreated or treated with the glycosidase PNGase F, which cleaves N-linked oligosaccharides from glycoproteins. As shown in FIG. 10, when the CHO 6-18 antibody was treated with PNGase F (left, lane 3), there was a molecular weight shift in the heavy chain of the IgG antibody. The treated heavy chain (left, lane 3) migrates at a lower molecular weight as compared to the heavy chain of the untreated antibody (left, lane 2), indicating the removal of the N-linked oligosaccharides. In the right panel, the 6-18 mouse mAb was either untreated (lane 2) or treated (lane 3) with the same PNGase F as the CHO 6-18 antibody. No molecular weight shift in the heavy chain and/or light chain was observed, suggesting that the 6-18 mouse mAb is not glycosylated or is glycosylated to a lesser extent than the CHO 6-18 antibody.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the protein constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where embodiments or aspects of the disclosure, is/are referred to as comprising particular elements, features, etc., certain embodiments or aspects consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification.

All patents, patent applications, websites, other publications or documents, accession numbers and the like cited herein are incorporated by reference in their entirety for all purposes to the same extent as if each individual item were specifically and individually indicated to be so incorporated by reference.

1. A method of detecting a cerebrospinal fluid (CSF) leakage in a subject, said method comprising:
a1) contacting a rhinorrhea or otorrhea sample from the subject with a first anti-tau antibody that binds to a first epitope of a human tau protein, wherein the first epitope comprises the amino acid sequence of QEFEVMEDHAGTY (SEQ ID NO: 1), and a second anti-tau antibody that binds to a second epitope of the human tau protein, wherein the second epitope comprises the amino acid sequence of AAPPGQKGQANA (SEQ ID NO: 2); and
b1) detecting the presence or absence of at least one tau protein in the rhinorrhea or otorrhea sample from the subject, wherein the at least one tau protein binds to both the first anti-tau antibody and the second anti-tau antibody, and wherein detecting the presence of the at least one tau protein in the rhinorrhea or otorrhea sample indicates that the subject has the CSF leakage and detecting the absence of the at least one tau protein in the rhinorrhea or otorrhea sample indicates that the subject does not have the CSF leakage; or
a2) contacting a rhinorrhea or otorrhea sample from the subject with a first anti-tau antibody and a second anti-tau antibody, where the first and second anti-tau antibodies bind to at least one tau fragment, wherein the at least one tau fragment comprises at least amino acids 6-168 of SEQ ID NO: 7; and
b2) detecting the presence or absence of at least one tau protein in the rhinorrhea or otorrhea sample from the subject, wherein detecting the presence of the at least one tau protein in the rhinorrhea or otorrhea sample indicates that the subject has the CSF leakage and detecting the absence of the at least one tau protein in the rhinorrhea or otorrhea sample indicates that the subject does not have the CSF leakage.

2. (canceled)

3. The method of claim 1, wherein the CSF leakage in the subject is caused by head trauma, spine injury, skull base defects, high pressure intracranial hydrocephalus, intracranial hypertension, traumatic brain injury (TBI), or concussion.

4-5. (canceled)

6. The method of claim 1, wherein:
a) the rhinorrhea or otorrhea sample from the subject is in a volume of from about 50 microliters to about 200 microliters and/or
b) the presence or absence of the at least one tau protein in the rhinorrhea or otorrhea sample from the subject is detected in about 1 minute to about 20 minutes after the contacting step.

7. (canceled)

8. A method of diagnosing a traumatic brain injury (TBI) or concussion in a subject in need thereof, said method comprising:
a1) contacting a biological sample from the subject with a first anti-tau antibody that binds to a first epitope of a human tau protein, wherein the first epitope comprises the amino acid sequence of QEFEVMEDHAGTY (SEQ ID NO: 1) and a second anti-tau antibody that binds to a second epitope of the human tau protein, wherein the second epitope comprises the amino acid sequence of AAPPGQKGQANA (SEQ ID NO: 2); and
b1) detecting the presence or absence of at least one tau protein in the biological sample from the subject, wherein the at least one tau protein binds to both the first anti-tau antibody and the second anti-tau antibody, and wherein detecting the presence of the at least one tau protein in the biological sample indicates that the subject has the TBI and detecting the absence of the at least one tau protein in the biological sample indicates that the subject does not have the TBI;
or
a2) contacting biological sample from the subject with a first anti-tau antibody and a second anti-tau antibody, where the first and second anti-tau antibodies bind to at least one tau fragment, wherein the at least one tau fragment comprises at least amino acids 6-168 of SEQ ID NO: 7; and
b2) detecting the presence or absence of at least one tau protein in the biological sample from the subject, wherein detecting the presence of the at least one tau protein in the biological sample indicates that the subject has the TBI and detecting the absence of the at least one tau protein in the biological sample indicates that the subject does not have the TBI.

9-10. (canceled)

11. The method of claim 8, wherein:
a) the biological sample is obtained from a nose or an ear of the subject;
b) the biological sample from the subject is in a volume of from about 50 microliters to about 200 microliters; and/or
c) the presence or absence of the at least one tau protein in the biological sample from the subject is detected in about 1 minute to about 20 minutes after the contacting step.

12-14. (canceled)

15. The method of claim 1, wherein:
a) the first anti-tau antibody comprises a light chain comprising an amino acid sequence having at least 90% sequence identity to SEQ ID NO: 3 and a heavy chain comprising an amino acid sequence having at least 90% sequence identity to SEQ ID NO: 4; and/or
b) the second anti-tau antibody comprises a light chain comprising an amino acid sequence having at least 90% sequence identity to SEQ ID NO: 5 and a heavy chain comprising an amino acid sequence having at least 90% sequence identity to SEQ ID NO: 6.

16-22. (canceled)

23. The method of claim 1, wherein:
a) the at least one tau protein is a fragment or isoform of tau having a molecular weight of from about 15 kDa to about 75 kDa;
b) the at least one tau protein is a fragment or isoform of tau having a molecular weight of from about 15 kDa to about 30 kDa;
c) the at least one tau protein is a fragment or isoform of tau having a molecular weight of about 17 kDa and/or about 30 kDa; or
d) the at least one tau protein to which the first and second anti-tau antibodies bind comprises a first tau fragment or isoform of about 17 kDa, a second tau fragment or isoform of about 25 kDa, a third tau fragment or isoform of about 30 kDa, a fourth tau fragment or isoform of about 35 kDa, a fifth tau fragment or isoform of about 50 kDa, and a sixth tau fragment or isoform of about 75 kDa.

24-28. (canceled)

29. The method of claim 1, wherein the method has a sensitivity of detecting the at least one tau protein in the rhinorrhea or otorrhea sample at a concentration of from about 0.3 pg/μl to about 1 pg/μl or from about 0.5 pg/μl to about 0.8 pg/μl.

30. (canceled)

31. A method of treating a subject in need thereof diagnosed with or suspected of having a traumatic brain injury (TBI), said method comprising:
a) diagnosing the subject as having a traumatic brain injury by the method of claim 1; and
b) administering to the subject a therapeutically effective amount of treatment for the TBI,
wherein the treatment for the TBI can comprise one or more of:
i) resting;
ii) administering to the subject a therapeutically effective amount of a pain reliever, an anti-seizure drug, a coma-inducing drug, and/or a diuretic;
iii) surgery; and
iv) rehabilitation.

32. (canceled)

33. A device comprising:
a) a non-immobilized antibody area;
b) a lateral flow membrane downstream of and configured for fluid communication with the non-immobilized antibody area;
c) a first anti-tau antibody that binds to a first epitope of the human tau protein, wherein the first epitope comprises the amino acid sequence of QEFEVMEDHAGTY (SEQ ID NO: 1); and
d) a second anti-tau antibody that binds to a second epitope of the human tau protein, wherein the second epitope comprises the amino acid sequence of AAPPGQKGQANA (SEQ ID NO: 2).

34. (canceled)

35. The device of claim 33, wherein the first anti-tau antibody is immobilized at a first location on the lateral flow membrane and the second anti-tau antibody is labeled and located in the non-immobilized antibody area.

36. The device of claim 33, further comprising:
a) a sample loading area upstream of and configured for fluid communication with the non-immobilized antibody area;
b) an absorbent area downstream of and configured for fluid communication with the lateral flow membrane;
c) a secondary antibody immobilized at a second location on the lateral flow membrane, wherein the secondary antibody is an anti-immunoglobulin antibody that binds to the second anti-tau antibody, and wherein the second location is optionally downstream of the first location; and/or
d) a non-specific antibody and/or a non-specific blocking agent, such as an IgG antibody.

37-40. (canceled)

41. The device of claim 33, wherein:
a) the second anti-tau antibody is labeled by conjugating to a colored latex particle, a gold nanoparticle, or a gold nano-shell; or
b) the second anti-tau antibody is labeled by conjugating to a gold nano-shell having an average diameter of from about 100 nm to about 200 nm.

42-44. (canceled)

45. The device of claim 33, wherein:
a) the sample loading area comprises a sample loading pad, wherein the sample loading pad comprises cellulose fibers or woven meshes;
b) the non-immobilized antibody area comprises a non-immobilized antibody pad, and wherein the non-immobilized antibody pad comprises glass fibers, cellulose fibers, surface-treated polyester filters, or surface-treated polypropylene filters;
c) the lateral flow membrane is a nitrocellulose membrane; and/or
d) the absorbent area comprises an absorbent pad, and wherein the absorbent pad comprises cellulose fibers or woven meshes.

46-48. (canceled)

49. The device of claim 33, further comprising:
a) a solid substrate supporting the sample loading area, the non-immobilized antibody area, the lateral flow membrane, and the absorbent area; and/or
b) a housing.

50. (canceled)

51. The device of claim 33, wherein:
a) the device is in form of a test strip, a dipstick, a flow through device, or a microfluidic device;
b) the device is a point-of-care diagnostic device; and/or
c) the device is an over-the-counter diagnostic device.

52. (canceled)

53. The device of claim 33, wherein:
a) the first anti-tau antibody comprises a light chain comprising an amino acid sequence having at least 90% sequence identity to SEQ ID NO: 3 and a heavy chain comprising an amino acid sequence having at least 90% sequence identity to SEQ ID NO: 4; and/or
b) the second anti-tau antibody comprises a light chain comprising an amino acid sequence having at least 90% sequence identity to SEQ ID NO: 5 and a heavy chain comprising an amino acid sequence having at least 90% sequence identity to SEQ ID NO: 6.

54-56. (canceled)

57. The device of claim 33, wherein the device has a sensitivity of detecting the at least one tau protein in a sample at a concentration of from about 0.3 pg/μl to about 1 pg/μl or from about 0.5 pg/μl to about 0.8 pg/μl.

58. (canceled)

59. The device of claim 33, wherein:
a) the device is configured to change color at the first location on the lateral flow membrane where the first anti-tau antibody is immobilized when the first and second anti-tau antibodies bind to at least one tau protein in a sample; and/or
b) the device is configured to change color at the second location on the lateral flow membrane where the secondary antibody is immobilized as a control to show that the labeled second anti-tau antibody has moved from the non-immobilized antibody area to the second location on the lateral flow membrane via lateral flow;
wherein the color change is visible to human eyes or detected by a lateral flow reader.

60-67. (canceled)

68. A method of diagnosing, or monitoring progression of recovery from, a concussion, traumatic brain injury, head trauma, spine injury, skull base defects, high pressure intracranial hydrocephalus, or intracranial hypertension in a subject in need thereof, said method comprising detecting the presence or absence of at least one tau protein in a biological sample from the subject using the device of claim 33, wherein optionally the biological sample is obtained from a nose or an ear of the subject.

69-77. (canceled)


SEQUENCE LISTING (7 sequences)

SEQ ID NO: 1 - protein, 13 aa, Homo sapiens
QEFEVMEDHAGTY

SEQ ID NO: 2 - protein, 12 aa, Homo sapiens
AAPPGQKGQANA

SEQ ID NO: 3 - protein, 219 aa, Mus musculus
DVLMTQTPLSLAVNLGDQASLSCRSSQTILHSNGNTYLEWYLQKPGQSPRLLIYKVSKRFSGVPDRFSGSGSGTDFTLKISRVEADDLGIYYCFQGSLVPWAFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 4 - protein, 441 aa, Mus musculus
EVQLVESGEDLVKPGGSLKLSCVASGFAFSSYGMSWVRQTPDMRLEWVATISSSGSRTYFPDSVKGRLTISRDNDKNILYLQMSSLRSEDTAMYYCTITWDGAMDYWGRGISVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 5 - protein, 218 aa, Mus musculus
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNE

SEQ ID NO: 6 - protein, 443 aa, Mus musculus
QVTLKESGPGILQPSQTLSLTCSFSGFSLSTFGMGVGWFRQPSGKGLEWLAHIWWDDDKYYNPALKSRLTISKDTSKNQVFLKIANVDTADTATYYCARNYDYDVFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 7 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL